(19) United States
(12) Patent Application Publication
(10) Pub. No.: US 2026/0199532 A1
(43) Pub. Date: Jul. 16, 2026

(54) RADIOLABELED COPPER CHELATES AND USE THEREOF

(71) Applicant: The Trustees of Indiana University, Bloomington, IN (US)

(72) Inventor: Mark Alan Green, Carmel, IN (US)

(21) Appl. No.: 19/450,426

(22) Filed: Jan. 15, 2026

Related U.S. Application Data
(60) Provisional application No. 63/745,723, filed on Jan. 15, 2025.

(51) Int. Cl. A61K 51/04; C07B 59/00; C07F 1/08

(52) U.S. Cl. CPC A61K 51/0478; C07B 59/004; C07F 1/08; A61K 2123/00; C07B 2200/05

(57) ABSTRACT

Disclosed herein are copper chelate compounds and their use in imaging methods, such as emission tomography (PET) imaging. The methods disclosed herein may be suitable for whole-body imaging.

### CROSS-REFERENCE TO RELATED APPLICATIONS

The present application claims priority to U.S. Provisional Patent Application No. 63/745,723, filed Jan. 15, 2025, the entire content of which is hereby incorporated by reference.

### STATEMENT REGARDING FEDERALLY SPONSORED RESEARCH OR DEVELOPMENT

Not applicable.

### BACKGROUND

Imaging to evaluate regional myocardial blood flow is routinely needed in the care of patients with coronary artery disease. Imaging to evaluate regional cerebral blood flow can be useful in connection with assessment of patients with a range of pathologies, including cerebrovascular disease, stroke, epilepsy, and dementia. Robust imaging to evaluate tumor perfusion is not routinely available at present but could be important in conjunction with independent imaging to assess tumor metabolism, and tumor response to treatment.

Because of the varied and distributed nature of metastatic disease, the clinical care of cancer patients often demands diagnostic methods that are compatible with whole-body imaging. Thus, whole-body scanning is a clinical standard in metabolic imaging of tumors with ¹⁸F-FDG, as well as in imaging to detect skeletal metastases based on rates of bone remodeling, and in the imaging of neuroendocrine tumors using somatostatin receptor-targeted agents, and the imaging of prostate cancer with prostate-specific-membrane-antigen (PSMA)-targeted agents. A technique for whole-body assessment of tumor perfusion would naturally complement metabolic imaging in the definition of the tumor microenvironment and could also assist in evaluation of patient response to antiangiogenic therapies.

The growth rates of solid tumors are often limited by their capacity to recruit vasculature for nutritive perfusion, leading to widespread interest in tumor vascular endothelium as a target for the action of chemotherapeutic drugs. Tumor vasculature is morphologically abnormal, and unlike normal tissues the rates of tumor perfusion and metabolism are often uncoupled and un-predictably heterogeneous. The heterogeneity of tumor perfusion affects the efficiency of drug delivery while also leading to zones of hypoxia that may be quite resistant to treatment by radiation therapy. A whole-body scanning technique for assessment of tumor perfusion would potentially complement standard whole-body ¹⁸F-FDG imaging of tumor metabolism, offering further insights into the physiology of a patient's tumors and their response to treatment.

¹⁵O-labeled water is the gold standard for PET quantification of regional tissue perfusion, having been validated to behave as a freely diffusible tracer in myocardium and to also robustly allow quantification of cerebral blood flow. As a freely diffusible tracer, ¹⁵O-water is also the standard for noninvasive imaging to quantify tumor perfusion. The 2-min physical half-life of ¹⁵O is ideally suited to imaging at high activity levels over the intrinsically brief time interval in which the distribution of a freely diffusible tracer is dominated by its perfusion-rate-limited delivery to tissue. However, for routine clinical imaging of tumor perfusion, ¹⁵O-water faces significant technical and practical barriers, including the need for an on-site cyclotron for ¹⁵O production; intrinsic need to image ¹⁵O-water at high counting rates to obtain good counting statistics; imaging being constrained to a single position of the scanner bed, precluding flow assessment using whole-body imaging protocols of the type routinely used in clinical oncology studies with ¹⁸F-FDG; and the need for invasive arterial blood sampling after each ¹⁵O-water injection, if flow is to be quantified and the heart, or another large arterial blood pool region, falls outside the camera's field of view.

There remains a need for agents and methods that allow for quantitative mapping of regional tissue perfusion and are suitable for use in whole-body imaging.

### SUMMARY

The present disclosure describes novel copper chelate compounds and their use in imaging methods, such as emission tomography (PET) imaging.

In one aspect, the present disclosure provides a compound of formula (I) or a pharmaceutically acceptable salt thereof,

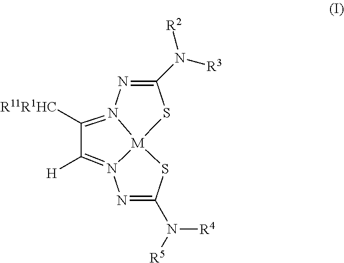

wherein
(i) R¹ is R^{A}—O—(CH₂)ₙ and R¹¹ is H or C₁₋₆alkyl; or
(ii) R¹ and R¹¹ together with the carbon atom they are attached to form a C₃₋₆cycloalkyl;
and
R², R³, R⁴, R⁵ are independently H or C₁₋₆alkyl;
R^{A} is H or C₁₋₆alkyl;
n is 0, 1, 2, 3, 4, 5, or 6; and
M is a copper isotope;
provided that R¹R¹¹CH— is not Me(EtO)CH.

In one aspect, the present disclosure provides a pharmaceutical composition comprising the compound as described herein, or a pharmaceutically acceptable salt thereof, and a pharmaceutically acceptable carrier.

In one aspect, the present disclosure provides a method of imaging a subject, the method comprising: administering the pharmaceutical composition as described herein to the subject; and acquiring a signal from the copper radioisotope in the subject, thereby generating an image of the subject.

In one aspect, the present disclosure provides a method of performing positron emission tomography (PET) imaging of a subject, the method comprising: administering the pharmaceutical composition as described herein to the subject; and acquiring a signal from the copper radioisotope in the subject by a gamma detector, thereby generating an image of the subject.

### BRIEF DESCRIPTION OF THE FIGURES

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows the sterile vial assembly (shown without shielding) configured for terminal sterilizing filtration, and subsequent withdrawal of the patient dose. Quality control (QC) samples are drawn from the vial before and/or after filling of the patient syringe, using the same side-port Luer-fitting as the patient dose syringe.

FIG. 2A shows the ⁶⁴Cu-Acetate Radiochromatogram-Silica Gel Plate eluted with ethanol. Origin at 21-mm. Solvent Front at 139-mm.

FIG. 2B shows the ⁶⁴Cu-Acetate Radiochromatogram-ITLC-SG strip eluted with ethanol.

Origin at 50-mm. Solvent Front at 133-mm.

FIG. 3A shows the ⁶⁴Cu-Acetate Radiochromatogram-Silica Gel Plate eluted with 5% saline in ethanol. Origin at 21-mm. Solvent Front at 134-mm.

FIG. 3B shows the ⁶⁴Cu-Acetate Radiochromatogram-ITLC-SG strip eluted with 5% saline in ethanol. Origin at 50-mm. Solvent Front at 135-mm.

FIG. 4A shows the ⁶⁴Cu-MeOPTSM Radiochromatogram-Silica Gel Plate eluted with ethanol. Origin at 20-mm. Solvent Front at 128-mm. (Product R_{f}=0.75.)

FIG. 4B shows the ⁶⁴Cu-MeOPTSM Radiochromatogram-ITLC-SG strip eluted with ethanol. Origin at 45-mm. Solvent Front at 127.5-mm. (Product R_{f}=0.73)

FIG. 5 shows the images of the Cu-MeOPTSM bulk material (top) and selected crystal mounted on Kapton loop (bottom).

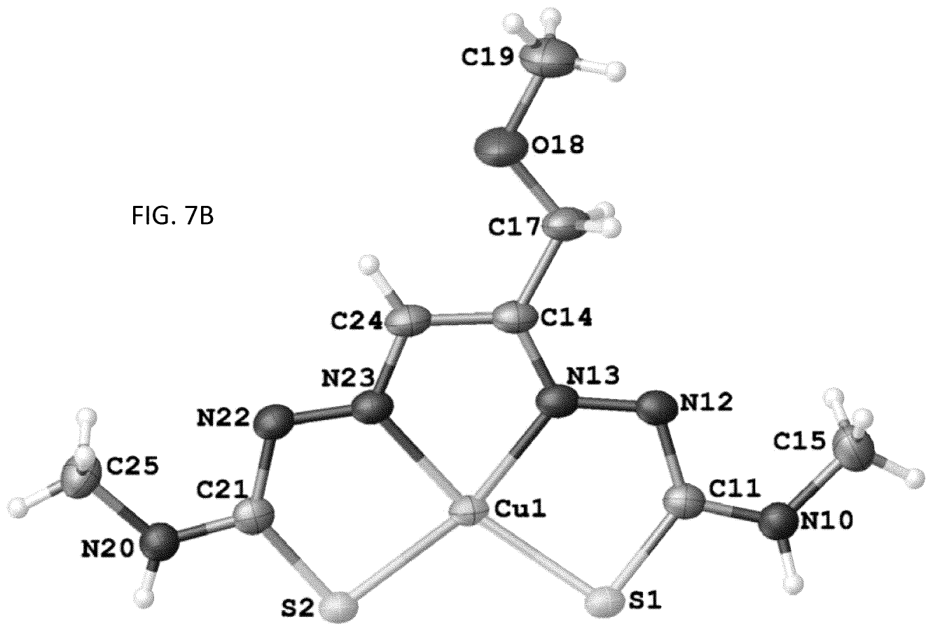

FIG. 6 shows the Cu-MeOPTSM formula unit as determined by X-ray crystallography.

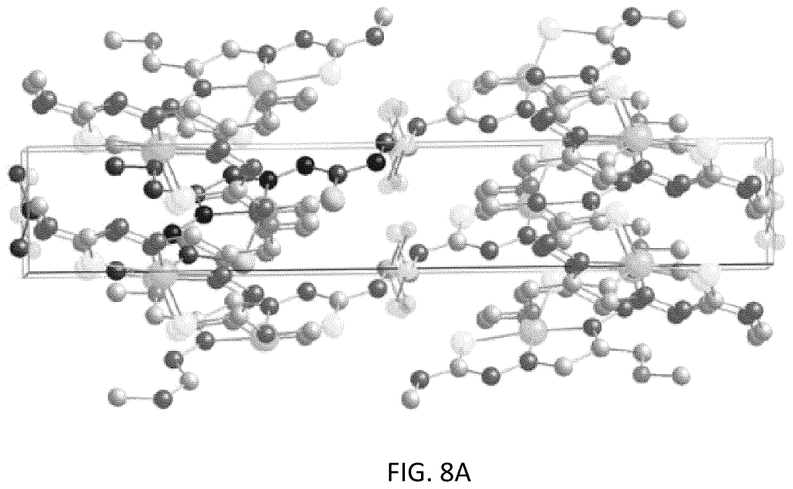

Disorder of the CH₂OCH₃ groups and disordered solvent EtOH are shown.

FIG. 7A shows the Cu complex, Cu-MeOPTSM, as determined by X-ray crystallography, disorder omitted.

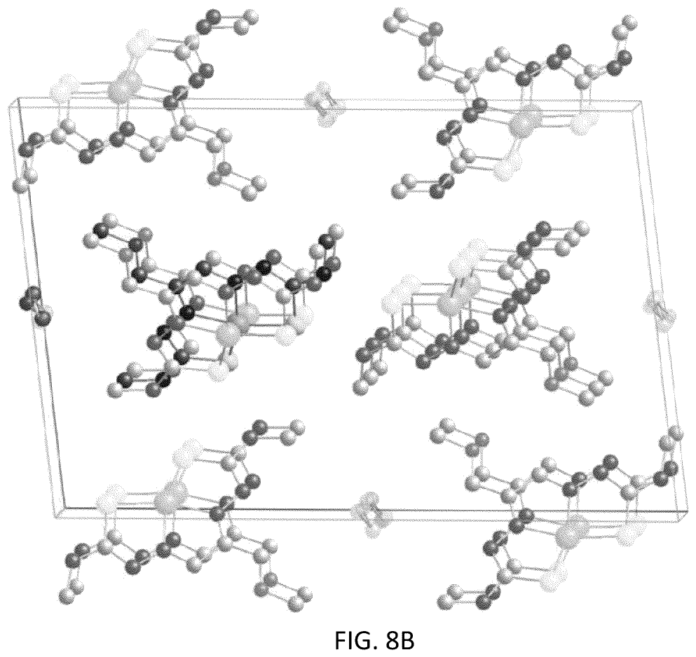

FIG. 7B shows the Cu complex, Cu-MeOPTSM, as determined by X-ray crystallography, (disorder omitted) with the atom numbering scheme employed in tabulation of the observed bond distances and angles.

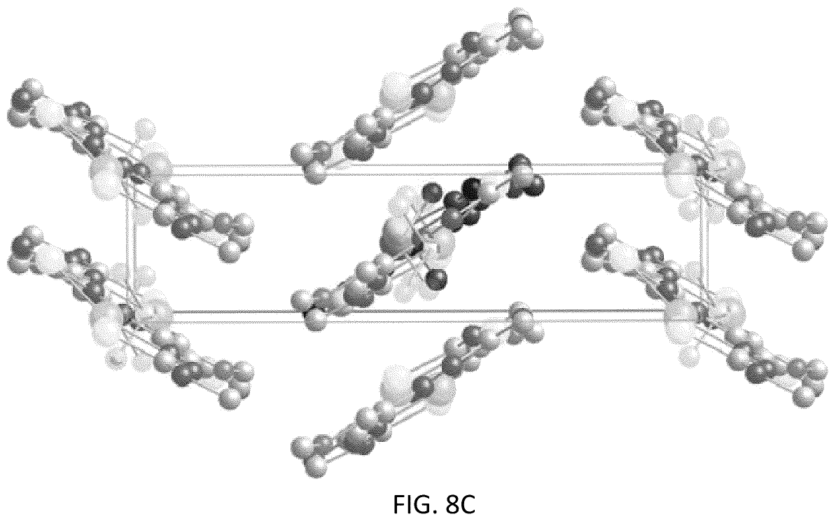

FIG. 8A shows the Cu-MeOPTSM cell plot as determined by X-ray crystallography, with view along a.

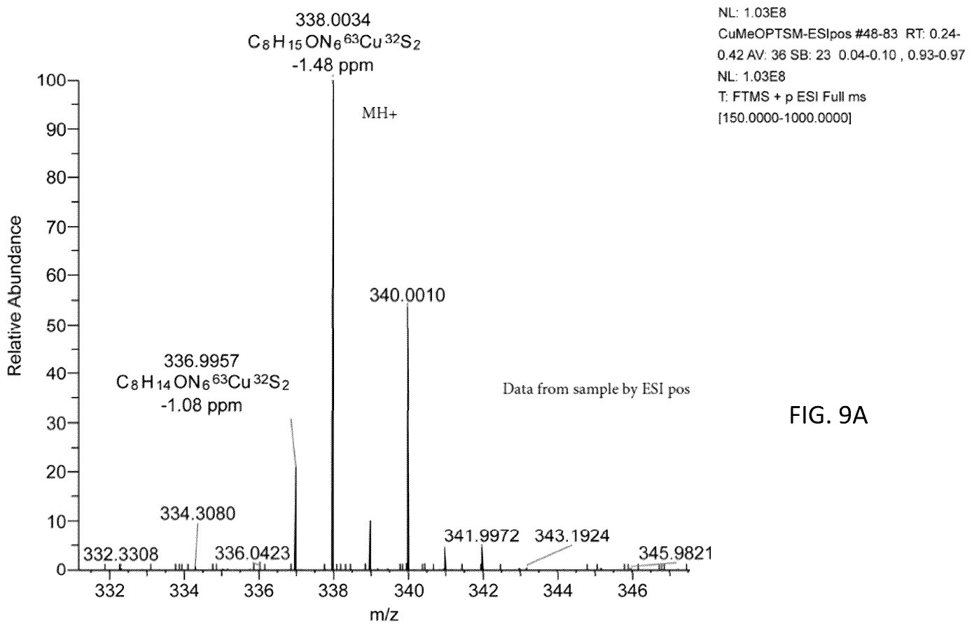

FIG. 8B shows the Cu-MeOPTSM cell plot as determined by X-ray crystallography, with view along b.

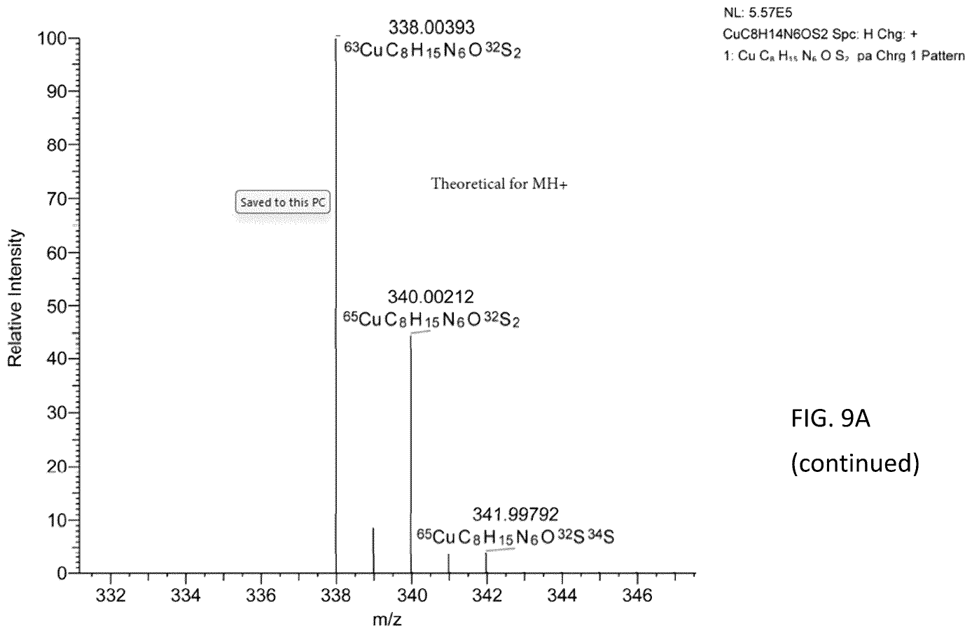

FIG. 8C shows the Cu-MeOPTSM cell plot as determined by X-ray crystallography, with view along c.

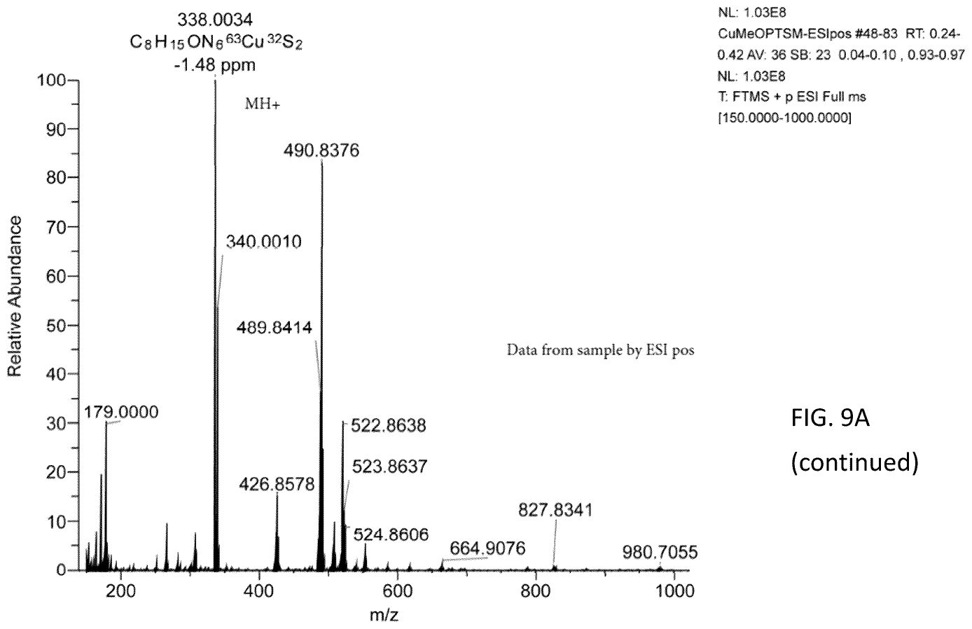

FIG. 9A shows the high resolution mass spectrometry of Cu-MeOPTSM.

FIG. 9B shows the predicted mass spectrum of Cu-MeOPTSM.

### DETAILED DESCRIPTION

Disclosed herein are metal chelate compounds, such as copper radiopharmaceuticals, that are suitable for administration to a subject in medical care and related imaging methods. The disclosed compounds, compositions, and methods may be suitable for whole-body imaging. The disclosed compounds, compositions, and methods may also be suitable for quantitative mapping of regional tissue perfusion.

Chemical entities and compounds of the present disclosure may be described using terms known in the art and defined herein.

The term “alkyl” as used herein refers to a monovalent saturated straight or branched hydrocarbon, such as a straight or branched group of 1-20, 1-12, 1-10, or 1-6 carbon atoms, referred to herein as C₁-C₂₀ alkyl (or C₁₋₂₀alkyl), C₁-C₁₂ alkyl (or C₁₋₁₂alkyl), C₁-C₁₀ alkyl (or C₁₋₁₀alkyl), or C₁-C₆ alkyl (or C₁₋₆alkyl), respectively.

The term “alkylene” refers to a divalent saturated straight or branched hydrocarbon group, such as a straight or branched group having 1-20, 1-12, 1-10, or 1-6 carbon atoms, referred to herein as a C₁-C₂₀ alkylene (or C₁₋₂₀alkylene), C₁-C₁₂ alkylene (or C₁₋₁₂alkylene), C₁-C₁₀ alkylene (or C₁₋₁₀alkylene), or C₁-C₆ alkylene (or C₁₋₆alkylene), respectively. An exemplary alkylene group is —CH₂CH₂—.

The term “alkenyl” refers to a monovalent straight or branched hydrocarbon group having one or more double bonds. An alkenyl group having up to 20 carbon atoms is referred to as a C₂-C₂₀ alkenyl (or C₂₋₂₀alkenyl). Likewise, for example, an alkenyl having up to 6 carbon atoms is referred to as a C₂-C₆ alkenyl (or C₂₋₆alkenyl).

The term “alkenylene” refers to a divalent straight or branched hydrocarbon group having one or more double bonds. An alkenylenyl group having up to 20 carbon atoms is referred to as a C₂-C₂₀ alkenylene (or C₂₋₂₀alkenylene). Likewise, for example, an alkenylene having up to 6 carbon atoms is referred to as a C₂-C₆ alkenylene (or C₂₋₆alkenylene).

The term “alkoxy” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “carboxy” or “carboxyl” as used herein refers to the group-COOH or its corresponding salts, e.g. —COONa, etc.

The term “aryl” is art-recognized and refers to a carbocyclic aromatic group. Representative aryl groups include phenyl, naphthyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls.

The term “arylene” refers to a divalent carbocyclic aromatic group. Representative arylene groups include-C₆H₄—, —C₁₀H₆—, and the like. The term “arylene” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls.

The term “phenyl” refers to a mono-substituted benzene ring and has a formula of —C₆H₅.

The term “heteroaryl” is art-recognized and refers to a heterocyclic aromatic group. Representative heteroaryl groups include pyridinyl, quinolinyl, furanyl, thionyl, and the like. The term “heteroaryl” includes polycyclic ring systems having two or more heterocyclic rings in which two or more carbon or heteroatom are common to two adjoining rings (the rings are “fused rings”) wherein at least one of the rings is a heterocyclic aromatic group and, e.g., the other ring(s) may be cycloalkyls, cycloalkenyls, cycloalkynyls, and/or aryls. In certain embodiments, the heteroaryl group is a 6-10 membered ring structure. The term “pyridyl” refers to a group derived from pyridine by removal of a hydrogen atom from a ring carbon atom in pyridine. The pyridyl group has a formula —C₅H₄N.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” or “C₄₋₈cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido or carboxyamido, amidino, amino, aryl, arylalkyl, azido, carbamate, carbonate, carboxy, cyano, cycloalkyl, ester, ether, formyl, halo, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a divalent saturated cyclic, bicyclic, or bridged cyclic (e.g., adamantyl) hydrocarbon group having, for example, 3-12, 3-8, 4-8, or 4-6 carbons derived from a cycloalkane. An exemplary cycloalkylene group is —C₃H₄—. Unless specified otherwise, cycloalkylene groups are optionally substituted at one or more ring positions with, for example, alkanoyl, alkoxy, alkyl, haloalkyl, alkenyl, alkynyl, amido or carboxyamido, amidino, amino, aryl, arylalkyl, azido, carbamate, carbonate, carboxy, cyano, cycloalkyl, ester, ether, formyl, halo, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkylene group is not substituted, i.e., it is unsubstituted.

The terms “heterocycloalkyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cₓ-C_{y} or C_{x-y} nomenclature where x and y are integers specifying the number of ring atoms. For example, a C₃-C₇ (or C₃₋₇) heterocyclyl group refers to a saturated or partially unsaturated 3- to 7-membered ring structure containing one to four heteroatoms, such as nitrogen, oxygen, and sulfur. The designation “C₃-C₇” or “C₃₋₇” indicates that the heterocyclic ring contains a total of from 3 to 7 ring atoms, inclusive of any heteroatoms that occupy a ring atom position. In one embodiment, the heterocyclyl is piperidinyl.

The term “halogen” refers to halogen atoms F, Cl, Br, and I, or halogen substituents fluoro (—F), chloro (—Cl), bromo (—Br), and iodo- (—I).

The term “haloalkyl” is art-recognized and refers to an alkyl group, as defined above, having halogen atoms, as defined above, replacing one or more hydrogen atoms. Representative haloalkyl groups include trifluoromethyl, dibromoethyl, monochloropropyl, and the like.

The term “hydroxy” refers to a group of the form —OH.

The term “hydroxyalkyl” refers to an alkyl, as defined herein, in which a hydrogen atom is replaced by —OH. Representative examples of hydroxyalkyl include, but are not limited to those derived from C₁₋₆ alkyls, such as —CH₂OH, —CH₂CH₂OH, —CH₂CH₂CH₂OH, and the like.

The term “nitro” refers to a group of the form —NO₂.

The term “cyano” refers to a group of the form —CN.

Terms such as “alkyl,” “cycloalkyl,” “alkylene,” “cycloalkylene,” etc. may be preceded by a designation indicating the number of atoms present in the group in a particular instance (e.g., “C₁₋₄alkyl”, “C₁-C₄alkyl”, “C₃₋₆cycloalkyl”, “C₃-C₆cycloalkyl”, “C₁₋₄alkylene”, “C₁-C₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁-C₄” or “C₁₋₄” the members of the group that follows may have any number of carbon atoms falling within the recited range. A “C₁-C₄alkyl” or “C₁₋₄alkyl,” for example, is an alkyl group having from 1 to 4 carbon atoms, however arranged (i.e., straight chain or branched).

If a group is described as being “substituted”, a non-hydrogen substituent group is in the place of hydrogen on a carbon or nitrogen of that group. Thus, for example, a substituted alkyl is an alkyl in which at least one non-hydrogen group is in the place of a hydrogen on the alkyl. To illustrate, monofluoroalkyl is alkyl substituted with a fluoro group, and difluoroalkyl is alkyl substituted with two fluoro groups. It should be recognized that if there is more than one substitution on a substituent, each non-hydrogen group may be identical or different (unless otherwise stated). Substituent groups include, but are not limited to, halogen, ═O, ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

When a group is referred to as “unsubstituted” or not referred to as “substituted” or “optionally substituted”, it means that the group does not have any substituents. If a group is described as being “optionally substituted”, the group may be either (1) not substituted or (2) substituted. If a group is described as being optionally substituted with up to a particular number of non-hydrogen substituents, that group may be either (1) not substituted; or (2) substituted by up to that particular number of substituent groups or by up to the maximum number of substitutable positions on that group, whichever is less.

If substituents are described as being independently selected from a group, each substituent is selected independent of the other. Each substituent, therefore, may be identical to or different from the other substituent(s).

A person of ordinary skill in the art would be able to choose the substituents that fulfill the valency rules. For example, in a non-solvated or non-salt form of a compound, nitrogen typically has three bonds attached to it and oxygen typically has two bonds attached to it.

As used herein, “salt” refers to acid addition salts and basic addition salts. It may also refer to those salts that may be prepared in situ during the final isolation and purification of the present compounds.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### Composition

In an aspect of the current disclosure, a compound of formula (I), or a pharmaceutically acceptable salt thereof, is provided:

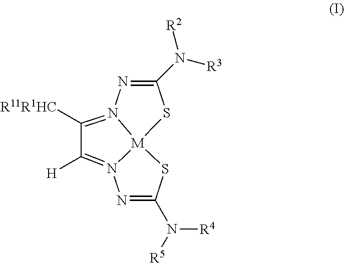

wherein
(i) R¹ is R^{A}—O—(CH₂)ₙ and R¹¹ is H or C₁₋₆alkyl; or
(ii) R¹ and R¹¹ together with the carbon atom they are attached to form a C₃₋₆cycloalkyl;
and
R², R³, R⁴, R⁵ are independently H or C₁₋₆alkyl;
R^{A} is H or C₁₋₆alkyl;
n is 0, 1, 2, 3, 4, 5, or 6; and
M is a copper isotope, such as a radioisotope of Cu selected from the group consisting of ⁶⁰C, ⁶¹Cu, ⁶²Cu, ⁶⁴Cu, and ⁶⁷Cu, or a stable nonradioactive isotope of Cu selected from the group consisting of ⁶³C and ⁶⁵Cu;
provided that R¹R¹¹CH— is not Me(EtO)CH.

In some embodiments,
R¹ is R^{A}—O—(CH₂)ₙ;
R¹¹, R², R³, R⁴, R⁵ are independently H or C₁₋₆alkyl;
R^{A} is H or C₁₋₆alkyl;
n is 0, 1, 2, 3, 4, 5, or 6; and
M is a copper radioisotope selected from the group consisting of ⁶⁰C, ⁶¹Cu, ⁶²Cu, ⁶⁴Cu, and ⁶⁷Cu,
provided that R¹R¹¹CH— is not Me(EtO)CH—.

In some embodiments, R¹¹ is H.

In some embodiments, R¹ and R¹¹ together with the carbon atom they are attached to form a cyclopropyl, cyclobutyl, or cyclopentyl.

In some embodiments, n is 0 or 1.

In some embodiments, R^{A} is H or methyl.

In some embodiments, R¹ is MeO—, MeOCH₂—, or HOCH₂—; and R¹¹ is H.

In some embodiments, R² and R⁴ are H; and R³ and R⁵ are C₁₋₆alkyl.

In some embodiments, R¹ is MeO— or MeOCH₂—; R¹¹ is H; R² and R⁴ are H; and R³ and R⁵ are methyl.

In some embodiments, M is ⁶¹Cu or ⁶²Cu or ⁶⁴Cu.

In some embodiments, M is ⁶²Cu or ⁶⁴Cu.

In some embodiments, M is ⁶³Cu or ⁶⁵Cu.

In some embodiments, the compound is

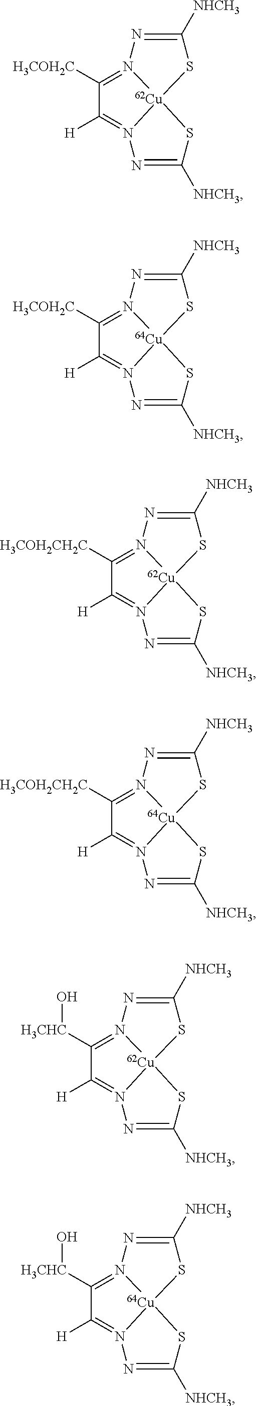

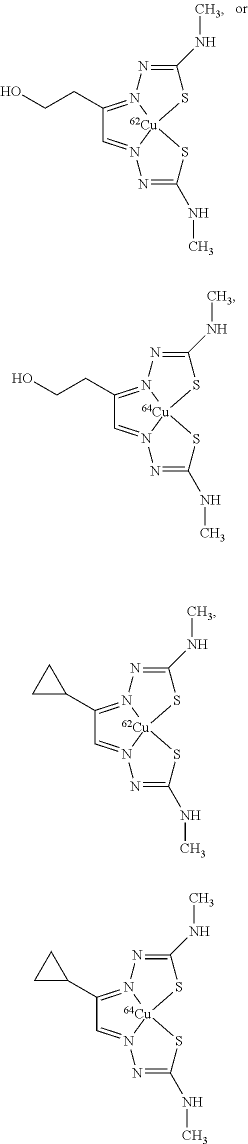

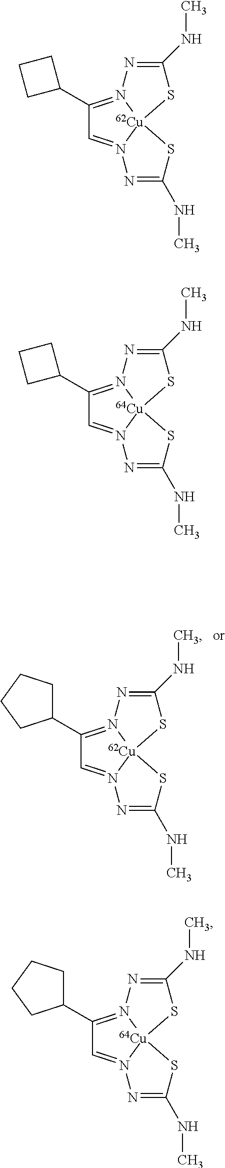

or a pharmaceutically acceptable salt thereof.

In some embodiments, the compound is

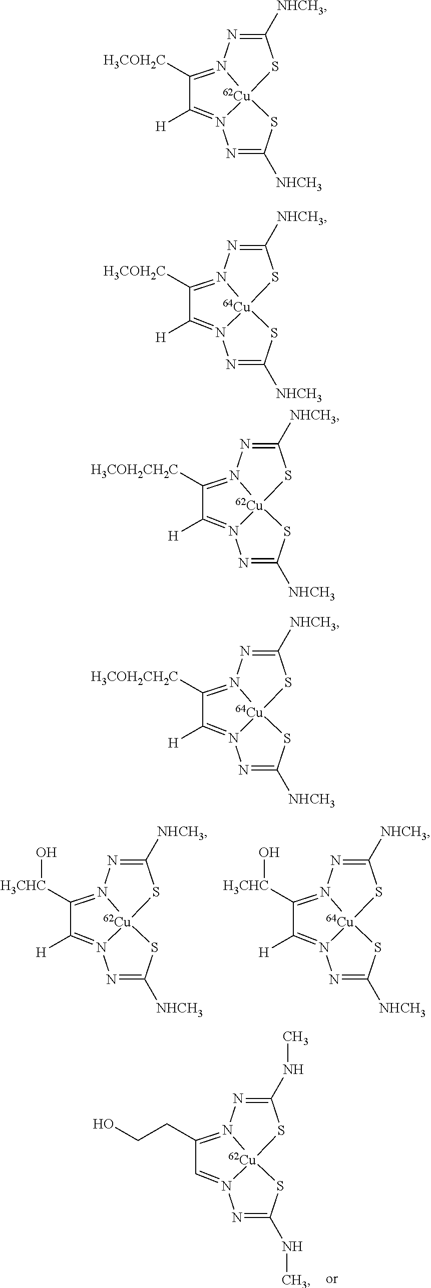

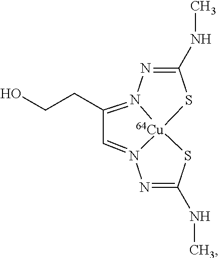

or a pharmaceutically acceptable salt thereof.

In some embodiments, the compounds disclosed herein may exhibit high first-pass extraction from blood into tissue after intravenous or intraarterial administration, which may assure that regional uptake maps the pattern of tissue perfusion. The compounds disclosed herein may also possess relatively nonspecific tissue retention of the radioactivity, which may allow time for acquisition of high-quality images based on external detection of their decay radiation.

### Pharmaceutical Compositions

In an aspect of the current disclosure, a pharmaceutical composition is provided. In some embodiments, the pharmaceutical composition comprises the compound disclosed herein, or a pharmaceutically acceptable salt thereof, and a pharmaceutically acceptable carrier. The pharmaceutical compositions may take any physical form which is pharmaceutically acceptable.

The compositions may contain from about 1-100 mCi (37-3700 MBq) of the compound in total, depending on the desired doses and the type of composition to be used. Larger quantities of radiocopper may also be employed, for example to obtain multiple patient doses of radiopharmaceutical drug product from a single production batch of the agent. The activity of the compounds employed in the compositions and methods disclosed herein are not believed to depend greatly on the nature of the composition, and, therefore, the compositions can be chosen and formulated primarily or solely for convenience and economy. In some embodiments, the pharmaceutical composition includes a compound as described herein in a range from about 10-30 mCi ⁶⁴Cu, or 5-30 mCi ⁶¹Cu, or 10-50 mCi ⁶²Cu per patient dose.

The term “pharmaceutically acceptable” means approved by a regulatory agency of the Federal or a state government or listed in the U.S. Examples of suitable pharmaceutical carriers are described in “Remington's Pharmaceutical Sciences” by E. W. Martin. Suitable pharmaceutically acceptable carriers include, but are not limited to, for example, suitable diluents, vehicles, excipients, preservatives, solubilizers, emulsifiers, liposomes, or nanoparticles, among others. Additionally, such pharmaceutically acceptable carriers may be aqueous or non-aqueous solutions, suspensions, and emulsions. Examples of nonaqueous solutions include propylene glycol, polyethylene glycol, vegetable oils such as olive oil, and injectable organic esters such as ethyl oleate. Aqueous carriers include isotonic solutions, hypertonic solutions, hypotonic solutions, alcoholic/aqueous solutions, emulsions or suspensions, including saline and buffered media. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The formulation should be selected according to the mode of administration. The compositions may include a pharmaceutical carrier, excipient, or diluent, which are nontoxic to the subject being exposed thereto at the dosages and concentrations employed. Examples of pharmaceutical carriers include buffers such as acetate, phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; sugar alcohols such as mannitol or sorbitol; salt-forming counterions such as sodium; and/or nonionic surfactants such as TWEEN brand surfactant, polyethylene glycol (PEG), and PLURONICS® surfactant.

Intravenous administration is an illustrative route of administering the compounds employed in the compositions and methods disclosed herein. Other illustrative routes of administration include intraarterial, transdermal, percutaneous, oral, intramuscular, intranasal, buccal, intrathecal, intracerebral, intratumoral, intraorgan, or intrarectal routes. The route of administration may be varied in any way, limited by the physical properties of the compounds being employed and the convenience and/or objectives of the subject and the caregiver. In some embodiments, the compounds employed in the compositions and methods disclosed herein may be administered through intra-tissue injection, such as to produce a high local tissue deposition and retention of the radiocopper. In some such embodiments, the radiocopper may become intracellularly trapped after diffusion across cell membranes into the thio-rich intracellular space, where the copper chelate (e.g., Cu-bis(thiosemicarbazone)) may be reduced leading to intracellular deposition of the radiolabel in the endogenous intracellular copper pool. For example, in some embodiments, a local cytotoxic radiation dose may be delivered to a subject using locally injected Cu-64 and/or Cu-67.

In some embodiments, the pharmaceutical composition disclosed herein is an injectable composition. In some such embodiments, the injectable composition may be suitable for intravenous administration.

Suitable formulations include those that are suitable for more than one route of administration. For example, the formulation can be one that is suitable for both oral and intravenous administration. For example, in some embodiments, the compounds employed in the compositions and methods disclosed herein may be administered through orally, such as through a timed-release oral formulation. In some such embodiment, incorporation of the copper agent may allow for depositing of an imageable trapped tissue marker, showing the release position in the GI tract. Alternatively, suitable formulations include those that are suitable for only one route of administration as well as those that are suitable for one or more routes of administration, but not suitable for one or more other routes of administration. For example, the formulation can be one that is suitable for oral, topical, transdermal, percutaneous, intravenous, intramuscular, intranasal, buccal, and/or intrathecal administration but not suitable for intracerebral administration.

The inert ingredients and manner of formulation of the pharmaceutical compositions may be selected from conventional technologies. The usual methods of formulation used in pharmaceutical science may be used here. Suitable types of compositions include, but are not limited to, solutions, parenteral solutions, and intranasal sprays. In some embodiments, the radiopharmaceutical composition as described herein is a sterile parenteral product solution suitable for intravenous or intraarterial administration.

The composition can be prepared with materials (e.g., actives, excipients, carriers (such as cyclodextrins), diluents, etc.) having properties (e.g., purity) that render the formulation suitable for administration to humans or non-human subjects. In some embodiments, the composition is suitable for use in humans. In some embodiments, the composition is prepared with materials having purity and/or other properties that render it suitable for administration to non-human subjects, but not suitable for administration to humans.

Each dosage unit may contain the dose of a given compound, for example, a daily or procedure dose, or each dosage unit may contain a fraction of the total dose, such as one-half or one-third of the dose. The amount of each compound to be contained in each dosage unit can depend, in part, on the identity of the particular compound chosen for the diagnosis or therapy and other factors, such as the indication for which it is given. The pharmaceutical compositions disclosed herein may be formulated so as to provide quick, sustained, or delayed release of the active ingredient after administration to the patient by employing well known procedures.

The composition may include a single compound or a combination of compounds as described herein for administration. For example, two or more of the compounds described herein may be included in the composition. In addition, the composition may include solvate forms of the compounds or salts, esters, and/or amides, thereof. Solvate forms may include ethanol solvates, hydrates, and the like.

The disclosed compounds or pharmaceutical compositions comprising the disclosed compounds may be administered with additional diagnostic or therapeutic agents. The additional agent may include, for example, one or more known radiopharmaceutical or imaging agents.

### Methods

In an aspect of the current disclosure, a method of imaging a subject is provided. The method comprises administering a pharmaceutical composition disclosed herein to the subject; and acquiring a signal from the copper radioisotope in the subject, thereby generating an image of the subject.

As used herein, a “subject” or a “subject in need thereof” may refer to a subject that is in need of imaging, such as medical imaging (e.g., PET, SPECT, or planar gamma imaging). The subject may have a disease or disorder. In some embodiments, the diseases or disorders include but are not limited to cerebrovascular diseases (e.g., transient ischemic attack (ITA) and stroke), cardiovascular disease, kidney disease, musculoskeletal disease or injury, cancer, dementia, Alzheimer's disease, and epilepsy. The subject may be human, or a non-human animal, e.g., a mammal, e.g., a horse, a dog, a cat, a goat, a donkey, etc.

In some embodiments, the signal is acquired by positron emission tomography (PET).

The pharmaceutical composition may be administered to the subject through different routes, such as through intravenous or intraarterial administration. In some embodiments, the pharmaceutical composition is administered to the subject via intravenous administration. For example, the composition may be formulated as an injectable composition.

The disclosed compound (e.g., copper radiopharmaceutical) may be synthesized as a sterile drug product suitable for intravenous administration by reacting the radioactive copper (II) ion with the bis(thiosemicarbazone) ligand. The reaction may be performed in an acetate-buffered saline-ethanol mixture suitable for direct intravenous administration, or the radiopharmaceutical agent could be prepared, trapped on a solid phase extraction cartridge for purification, and recovered in pure form with ethanol and formulated with saline and excipients suitable for intravenous injection.

In some embodiments, administering the pharmaceutical composition to the subject comprises perfusing a tissue of the subject with the compound disclosed herein, or a pharmaceutically acceptable salt thereof; and wherein the signal from the perfused tissue is acquired. In some embodiments, perfusing a tissue of the subject may include, but is not limited to, regional cerebral perfusion, myocardial perfusion, renal perfusion, muscle perfusion, and tumor perfusion.

Imaging of the tissue perfusion may be achieved qualitatively and/or quantitatively. For imaging with quantitation of perfusion, the compound or composition disclosed herein may be administered with the patient positioned in the PET camera for a dynamic data acquisition that allows quantification of the arterial input function from the observed time-activity curve for radiopharmaceutical in the left ventricular or atrial blood pool, or a region of interest defined to capture the radioactivity levels in a major artery. Whole body (or specific region-of-interest) imaging would then occur to map the tissue concentrations of the radioactivity. A tracer kinetic model may be applied for conversion of the resulting tissue images into perfusion maps in mL blood per minute per gram of tissue based on the observed tissue concentration(s) of radioactivity and the image-derived arterial input function characterizing the change in arterial concentration of the radiopharmaceutical over time. Conversion of the resulting PET images from radioactivity concentration into absolute perfusion maps in units of mL blood per minute per gram of tissue may be achieved, for example, using software such as Q-image developed by G. Hutchins in the Department of Radiology and Imaging Sciences.

In some embodiments, the image is generated from the whole body of the subject. Whole body imaging is important for applications in oncology, since many cancers will ultimately involve the appearance of metastatic tumors at multiple distributed sites within the body.

In some embodiments, the method further comprises quantitatively mapping the signal from one or more perfused tissues of the subject.

In some embodiments, the subject is a human.

In some embodiments, the method further comprises evaluating a physiological condition of the subject based on the image. The physiological condition may comprise myocardial blood flow, cerebral blood flow, renal blood flow, tumor blood flow, musculoskeletal blood flow, other organ blood flow, or a combination thereof. In some embodiments, evaluating a physiological condition of the subject comprises evaluating perfusion rates in other organs and tissues, such as in the muscle.

In some embodiments, the subject has a disease. In some embodiments, the disease may be cerebrovascular disease, stroke, epilepsy, dementia, cardiovascular disease, kidney disease, musculoskeletal disease or injury, cancer, or a combination thereof.

In an aspect of the current disclosure, a method of performing positron emission tomography (PET) imaging of a subject is provided. The method comprises administering a pharmaceutical composition disclosed herein to the subject, and acquiring a signal from the copper radioisotope in the subject by a radiation detector, such as a gamma detector, thereby generating an image of the subject.

In some embodiments, the image is generated from whole body of the subject.

In some embodiments, the subject has a disease. In some embodiments, the disease may be cerebrovascular disease, stroke, epilepsy, dementia, cancer, or a combination thereof.

In an aspect of the current disclosure, a method of performing autoradiography is provided, such as a method of identifying tissue-specific distribution of a radiolabeled complex comprising a radiocopper in a subject. The method comprises imaging an excised tissue having the radiocopper therein. Autoradiography is a molecular imaging technique used for the localization of radiolabeled compounds in specimens and can be used to study the local distribution of the radioactive isotopes.

In some embodiments, the method further comprises administering to the subject the pharmaceutical composition described herein, and excising the tissue from the subject after administrating the pharmaceutical composition. In some embodiments, the method further comprises excising the tissue from the subject, and contacting the pharmaceutical composition described herein with the excised tissue. The radiocopper of the radiolabeled complex may be detected and evaluated. In some embodiments, the radiolabeled complex comprises the compound of formula (I) and the radiocopper may comprise a copper radioisotope selected from the group consisting of ⁶⁰C, ⁶¹Cu, ⁶²Cu, ⁶⁴Cu, and ⁶⁷Cu. In some embodiments, the radiolabeled complex may not stay intact at the time of the autoradiography. For example, the radiocopper of the radiolabeled complex may be liberated from chelating ligands through reductive decomposition of the copper chelate, liberating ionic copper that is “trapped” in the endogenous intracellular copper pool.

### EXAMPLES

The following Examples are illustrative and should not be interpreted to limit the scope of the claimed subject matter.

### Example 1—Preparation of ⁶⁴Cu-MeOPTSM and ⁶⁴Cu-MeOETSM

Synthetic pathways towards ⁶⁴Cu-MeOPTSM or ⁶⁴Cu-MeOETSM are shown in Schemes 1 and 2. The synthetic approach is based on methods described in Green et al. (J Med Chem 40:132-136; 1997; J Med Chem 33:1764-1770, 1990; Applied Radiation and Isotopes 42:317-320; 1991), the content of which is incorporated herein by reference in its entirety.

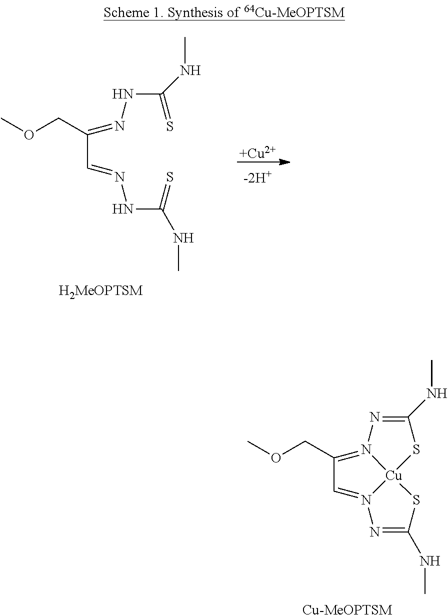

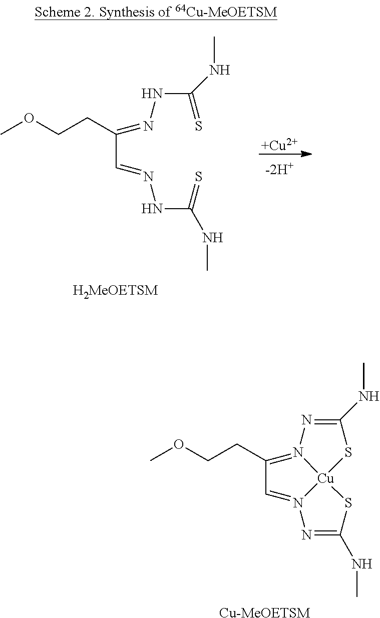

⁶⁴Cu Radionuclide: The ⁶⁴Cu²⁺ ion can be obtained in 0.2% HCl from Curium Pharma. The radionuclide is shipped from Curium for overnight delivery and is accompanied by a Certificate of Analysis documenting conformity to the release specifications of Curium's DMF for this radiopharmaceutical precursor. The solution nominally contains 70-110 mCi ⁶⁴Cu in 0.1-0.2 mL at the time of calibration (06:00 CT day of shipping.)

[⁶⁴Cu]Cu-MeOPTSM production: The ethylglyoxal bis(thiosemicarbazone) chelating ligand is chemically stable in ethanol solution. Because of the minute quantity of the H₂MeOPTSM ligand required per synthesis (15 μg) an ethanol stock solution of H₂MeOPTSM is prepared in a volumetric flask with an H₂MeOPTSM mass that is conveniently weighed, and the resulting stock solution stored at ~−5° C. The HCl solution of ⁶⁴Cu²⁺ radionuclide is buffered by addition of ultrapure sodium acetate followed by addition of the H₂MeOPTSM ligand in ethanol. The H₂MeOPTSM ligand avidly binds the Cu²⁺ ion, with the reaction rapidly going to completion in less than 5-minutes at room temperature. The uncharged, lipophilic [⁶⁴Cu]Cu-MeOPTSM product can be trapped by passing the reaction mixture through a conditioned C18-SepPak Light solid phase extraction cartridge, and then washed with sterile saline, recovered by elution with ethanol, and formulated to ≤5% ethanol by dilution with sodium chloride for injection. However, because the formation of [⁶⁴Cu]Cu-MeOPTSM with the high purity Curium ⁶⁴Cu proceeds with excellent yield, the solid-phase extraction step can be omitted to speed production and reduce personnel radiation exposure. Accordingly, the acetate-buffered ethanol solution of intermediate [⁶⁴Cu]Cu-MeOPTSM product can simply be diluted by addition of USP propylene glycol (to assure solubility of the lipophilic product), sterile water (USP), and sodium chloride for injection (USP), and then passed through a sterile 0.2-μm PVDF filter into a sterile evacuated vial to yield the final sterile [⁶⁴Cu]Cu-MeOPTSM drug product in acetate buffered saline with 5% ethanol and 5% propylene glycol.

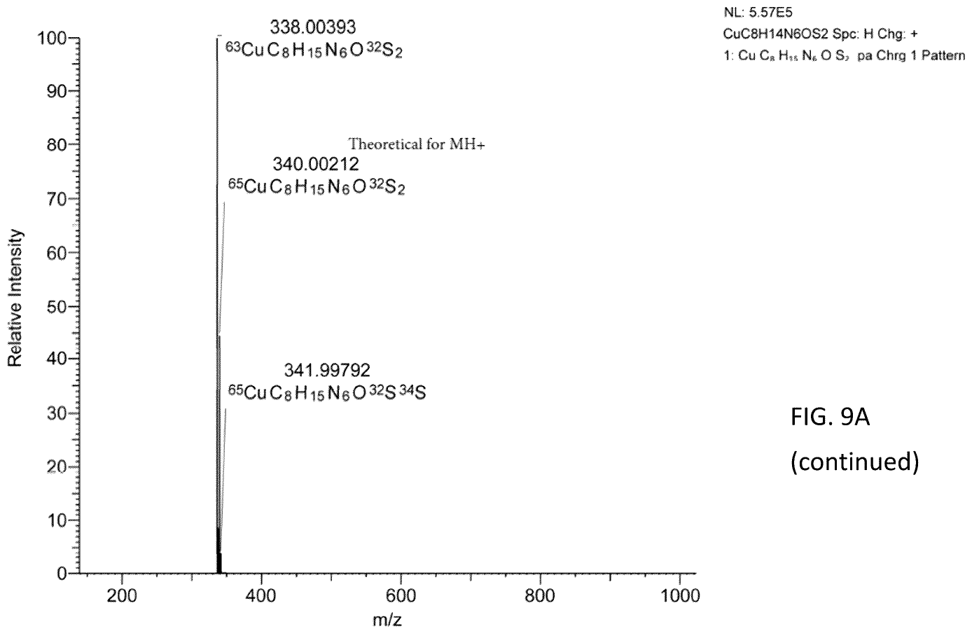

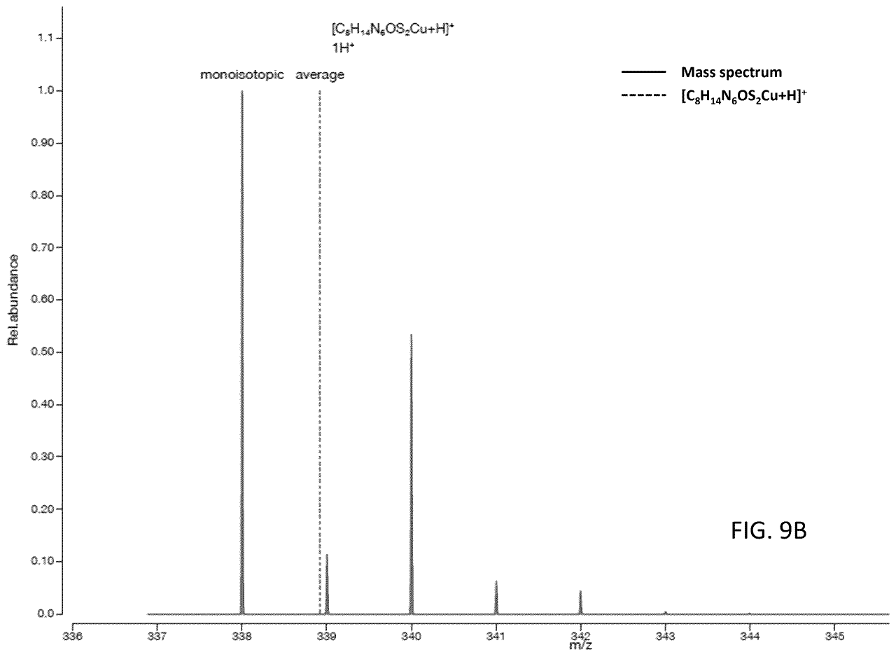

The preparation of Cu-MeOPTSM was confirmed by high resolution mass spectrometry (FIGS. 9A and 9B).

⁶⁴Cu-MeOETSM may be prepared in a similar fashion as ⁶⁴Cu-MeOPTSM.

The Standard Operating Procedure (SOP) for preparing ⁶⁴Cu-MeOPTSM or ⁶⁴Cu-MeOETSM is detailed below:

1. Check-in ⁶⁴Cu copper chloride shipment, completing supply information required on the Batch Record Form, and confirm that the material was tested to meet its established release specifications (shown on the associated Certificate of Analysis).

2. Condition the C18 SepPak Light (or C18 SepPak Vac) unit by passing through it 10-mL ethanol, followed by 10-mL sterile water for injection, leaving the unit wetted by the aqueous flush. (Optional-only necessary if solid phase extraction is to be employed in reformulation of [⁶⁴Cu]Cu-MeOPTSM or [⁶⁴Cu]Cu-MeOETSM product.)

3. Clean the interior of the laminar flow hood or biological safety cabinet that will be used during dispensing of the final sterile product.

4. To the ⁶⁴Cu-chloride (in the shipping vial, nominally 0.1-mL 0.2% HCl), add 20-100-μL 0.25M Sodium Acetate (ultrapure), followed by 10-15 μg H₂MeOPTSM or H₂MeOETSM dissolved in 0.3 mL absolute ethanol (USP). Gently swirl vial to mix, and also invert 5-times to assure any ⁶⁴Cu²⁺ on walls and septum have contacted the reaction solution, then allow to stand for at least 5-minutes. (Reaction to form [⁶⁴Cu]Cu-MeOPTSM or [⁶⁴Cu]Cu-MeOETSM is expected to be near instantaneous.) (The exact volume of the 0.25M sodium acetate solution can be adjusted as needed. Employing two equivalents of NaOAc per equivalent of HCl in the Cu-64 solution will raise the pH of the reaction solution to the buffer region for acetic acid/sodium acetate. In practice, simply adding the 0.25M NaOAc solution in a volume ~5 times the volume of the ⁶⁴CuCl₂ solution has been found to yield satisfactory results. The specification to employ ultrapure grade NaOAc is to minimize introduction of trace metal ion contamination into the reaction mixture.)

The total quantity of copper in the radiopharmaceutical product formulation will vary somewhat from batch to batch, since stable copper (⁶³Cu, ⁶⁵Cu) will be adventitiously present at variable trace levels in the various reagents. Due to the very small mass quantities of radiocopper present in radiopharmaceutical products, that adventitious stable Cu will typically be present at a higher concentration than the radiocopper isotope.

The Cu²⁺ ion and the bis(thiosemicarbazone) ligand react with a 1:1 stoichiometry in making the copper (II) bis(thiosemicarbazone) radiopharmaceutical. For optimal radiochemical purity, the quantity of the bis(thiosemicarbazone) ligand must be sufficient to efficiently bind all Cu²⁺ ions (stable plus radioactive) present in the formulation. Other trace metal contaminants (e.g., Ni²⁺) will compete with Cu²⁺ for chelation by the bis(thiosemicarbazone) ligand, but are expected to be only minor contaminants, and (except for potentially depleting available bis(thiosemicarbazone) ligand) will not interfere with the performance of the copper bis(thiosemicarbazone) pharmaceutical.

5. To directly formulate the final [⁶⁴Cu]Cu-MeOPTSM or [⁶⁴Cu]Cu-MeOETSM drug product, dilute resulting [⁶⁴Cu]Cu-MeOPTSM or [⁶⁴Cu]Cu-MeOETSM product to its final formulation by addition of 0.3 mL propylene glycol USP, 0.3 mL sterile water for injection, and 5.0 mL sodium chloride for injection. Total volume will be ~6-mL.

6. Draw the [⁶⁴Cu]Cu-MeOPTSM or [⁶⁴Cu]Cu-MeOETSM product solution into a shielded 12-mL sterile syringe, cap the syringe, then spray the outside of the shielded syringe with 70% sterile isopropanol to sanitize the surfaces, and take the shielded syringe into the clean laminar flow hood.

7. Inside the clean laminar flow hood or biological safety cabinet, attach a sterile 0.2-μm PVDF filter to the shielded [⁶⁴Cu]Cu-MeOPTSM or [⁶⁴Cu]Cu-MeOETSM syringe, and use a sterile 3-way stopcock to attach a sterile 3-inch (or longer) 18-21-gauge needle to the filter outlet. The side-port for the 3-way stopcock should remain capped, and the “OFF” designation of the valve should be pointed towards that capped side-port.

8. Sanitize the septum of a sterile evacuated 30-mL glass vial by swabbing with a sterile alcohol swab within the laminar flow hood or biological safety cabinet.

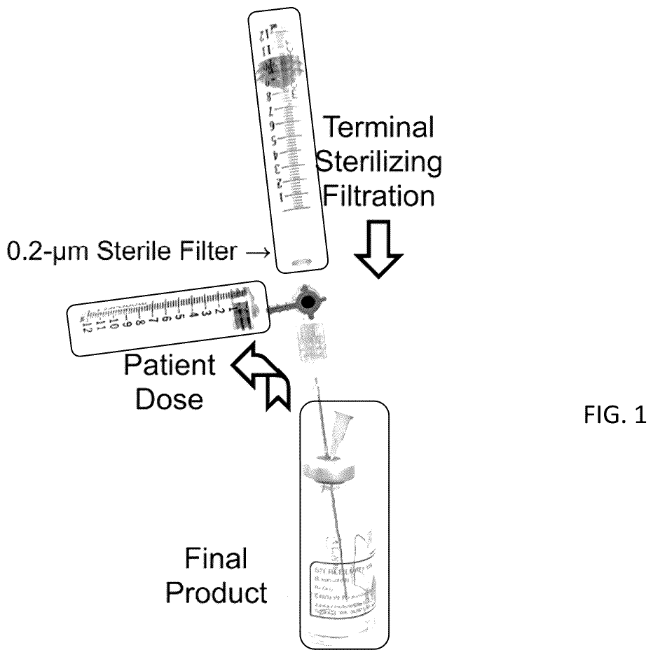

9. Pass the [⁶⁴Cu]Cu-MeOPTSM or [⁶⁴Cu]Cu-MeOETSM solution through the sterile 0.2-μm PVDF membrane filter, collecting the final [⁶⁴Cu]Cu-MeOPTSM or [⁶⁴Cu]Cu-MeOETSM product in the sterile septum-capped vial (FIG. 1).

10. Turn the “OFF” designation of the 3-way stopcock to point towards the sterilizing filter, and disconnect the filter and syringe from the stopcock. (Note: the filter MUST be retained for Bubble Point testing, below.) The stopcock-capped needle should be left in the septum, with the needle tip pushed to the bottom of the product vial. The still unused side-port of the 3-way stopcock must remain capped until that cap is replaced with the syringes for withdrawal of the final product quality control (QC) sample and the [⁶⁴Cu]Cu-MeOPTSM or [⁶⁴Cu]Cu-MeOETSM dose.

11. Pierce the product vial septum with a 23-gauge needle attached to a sterile filter, allowing the vial headspace to come to atmospheric pressure with the addition of sterile air.

12. Pull a 1-mL sample of the [⁶⁴Cu]Cu-MeOPTSM or [⁶⁴Cu]Cu-MeOETSM final product solution into the 1-mL syringe. This will be the material used for the destructive elements of quality control testing (assessment of radiochemical purity and pH; measurement of endotoxin level; and retrospective sterility testing).

13. Pull desired volume of [⁶⁴Cu]Cu-MeOPTSM or [⁶⁴Cu]Cu-MeOETSM final product solution into a sterile 10-12-mL syringe, and then cap the syringe with a sterile Luer cap.

14. Aseptically transfer 0.25-mL samples of the [⁶⁴Cu]Cu-MeOPTSM or [⁶⁴Cu]Cu-MeOETSM product to the Tryptic Soy Broth and Thioglycollate media vials and label with the [⁶⁴Cu]Cu-MeOPTSM or [⁶⁴Cu]Cu-MeOETSM product lot number.

15. Transfer an 0.3 mL aliquot of the [⁶⁴Cu]Cu-MeOPTSM or [⁶⁴Cu]Cu-MeOETSM product to a Endosafe vial containing 30-mL sterile water for dilution, then transfer the appropriate aliquots to an Endosafe-PTS LAL Cartridge for endotoxin testing.

16. Spot a ~1.0 μL drop of the [⁶⁴Cu]Cu-MeOPTSM or [⁶⁴Cu]Cu-MeOETSM product at the pre-marked “origin” on an ITLC-SG strip and immediately develop with ethanol (or 5% saline in ethanol), marking the location of the solvent front upon removing the ITLC-SG strip from the developing tank. (Alternatively, TLC analysis can be performed using a glass-backed silica gel TLC plate developed with ethanol. However, this may require >30-minutes to develop.)

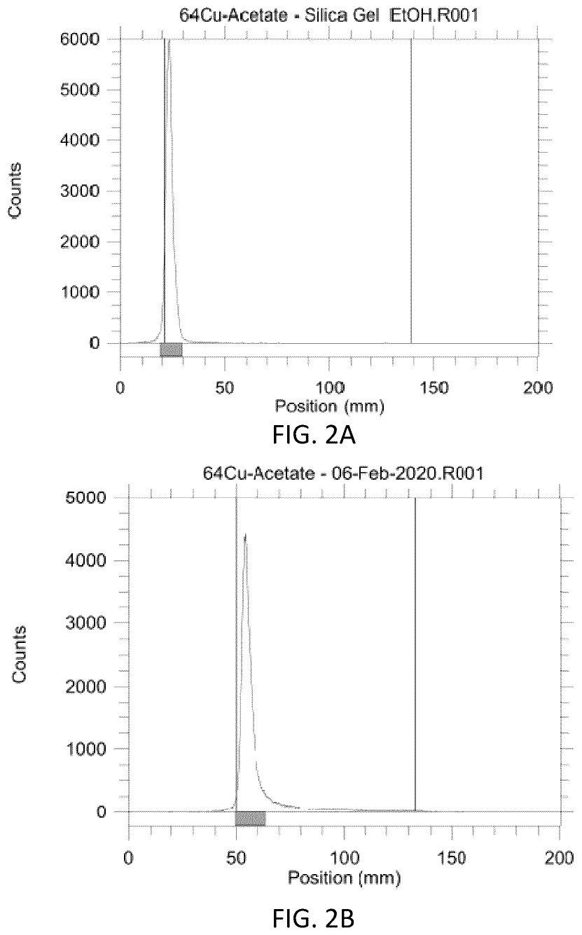

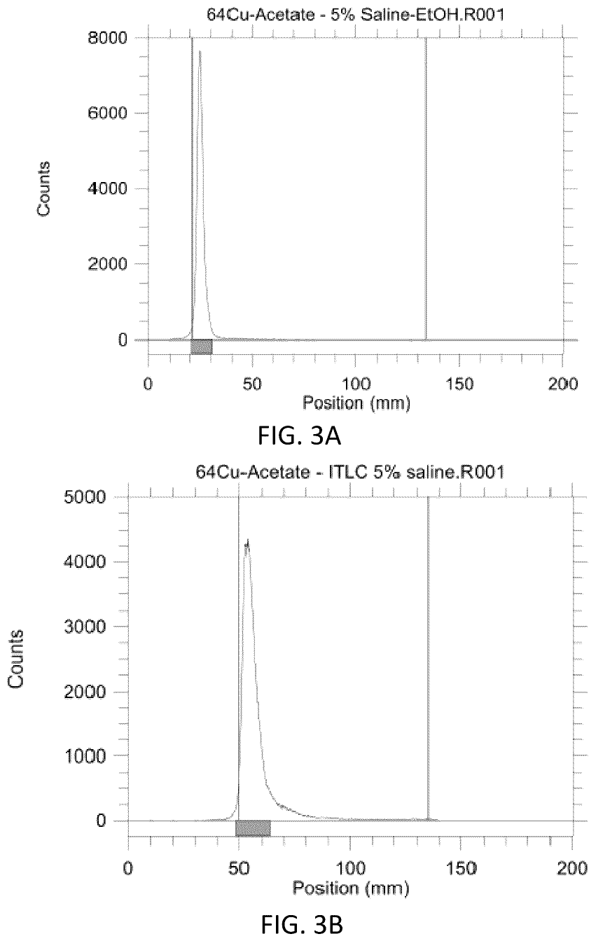

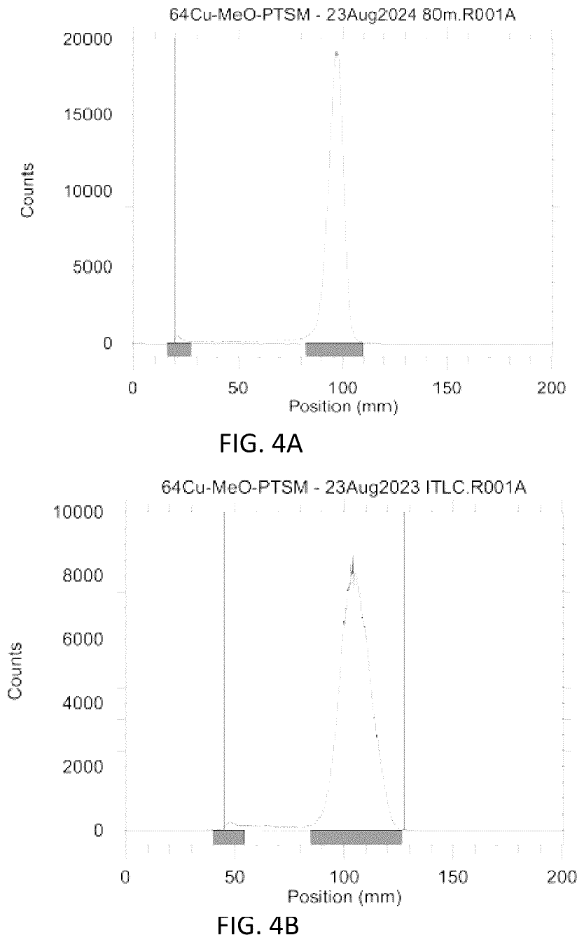

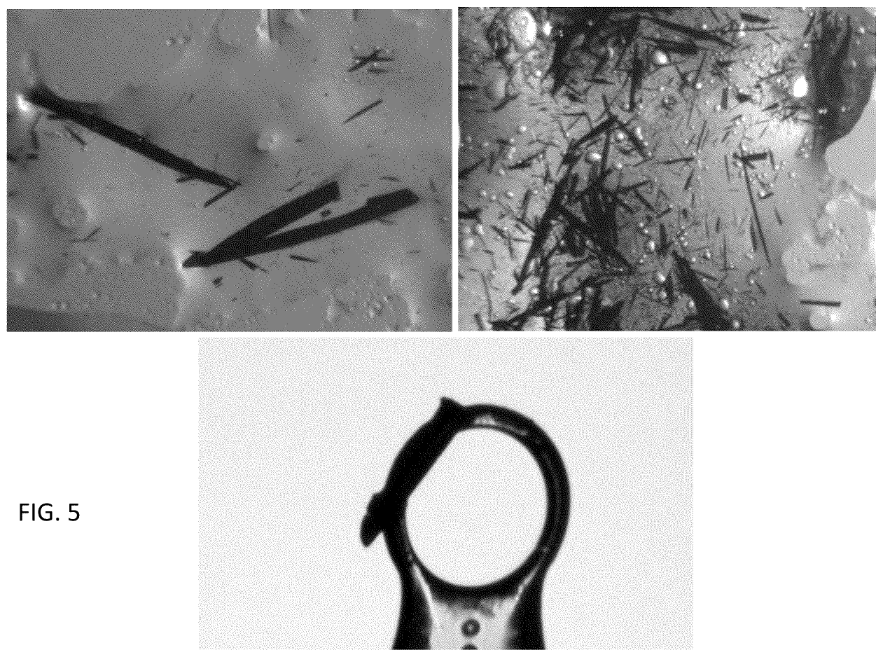

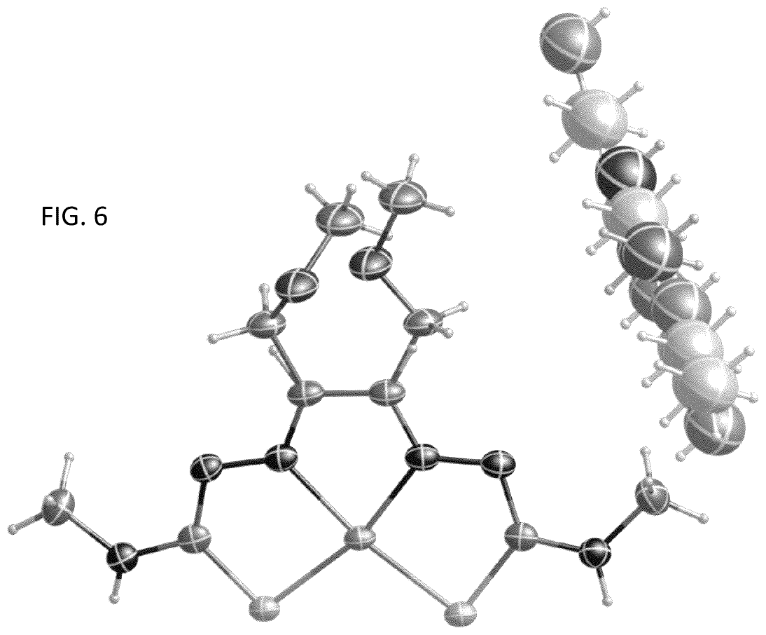

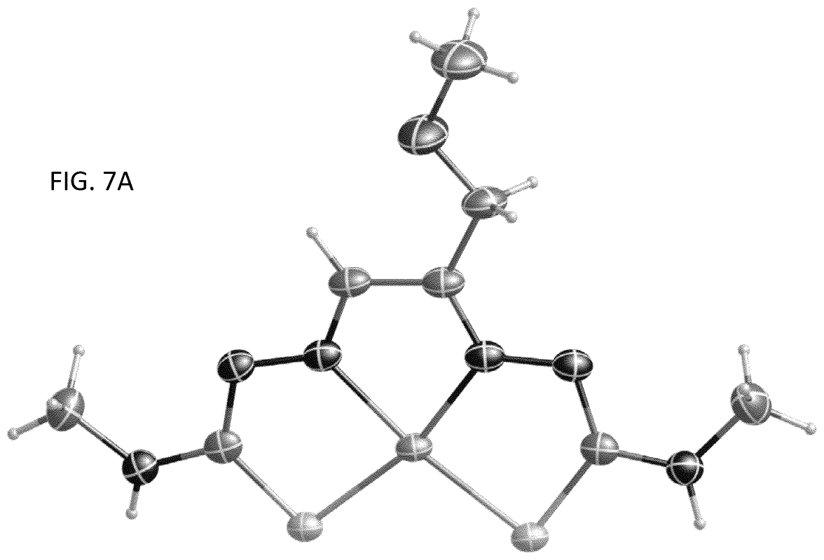

17. Cut the ITLC-SG strip at one-third the distance from the origin to the solvent front (R_{f}=0.33), and assay the ⁶⁴Cu on each piece with the dose calibrator or gamma counter. [The [⁶⁴Cu]Cu-MeOPTSM or [⁶⁴Cu]Cu-MeOETSM product will migrate with R_{f}=0.7-1.0 (top ⅔ of strip), while any unreacted ionic ⁶⁴Cu²⁺ will remain at the origin. See sample chromatograms in FIGS. 2A-2B, 3A-3B, 4A-4B.]

18. Calculate the purity of the [⁶⁴Cu]Cu-MeOPTSM or [⁶⁴Cu]Cu-MeOETSM product as:

$$Product Radiochemical Purity = \frac{\begin{matrix}Radioactivity Migrating on ITLC Strip \\ ( Upper or Solvent Front Segment )\end{matrix}}{\begin{matrix}Total Radioactivity on ITLC Strip \\ ( Origin Segment + Solvent Front Segment )\end{matrix}} \times 100 \%$$

19. Verify the pH of the final [⁶⁴Cu]Cu-MeOPTSM or [⁶⁴Cu]Cu-MeOETSM product using pH test strips.

20. Confirm the radionuclide identity for the ⁶⁴Cu by determining the half-life of the final product. (This determination will involve serial timed measurements of radioactivity in the sample over a 10-20-minute period.) The measured half-live should be within 5% of the 12.701-hour (762.1-minute) expected value (i.e., between 724.0 and 800.2 minutes). This test is required only for process validation batches. Otherwise, radionuclidic identity and purity are already adequately established as long as the Certificate of Analysis for the ⁶⁴Cu-chloride shipment has been confirmed to meet its established radionuclidic purity specifications. (Nothing in the [⁶⁴Cu]Cu-MeOPTSM or [⁶⁴Cu]Cu-MeOETSM production process can reduce the final product's radionuclidic purity relative to the starting ⁶⁴Cu.)

21. Complete the Batch Record and Dose Label, and release the [⁶⁴Cu]Cu-MeOPTSM or [⁶⁴Cu]Cu-MeOETSM final product if all pre-release tests are satisfactory.

22. If the [⁶⁴Cu]Cu-MeOPTSM or [⁶⁴Cu]Cu-MeOETSM radiopharmaceutical is being produced for “immediate use,” the labeled product expiration time will be no more than 60-minutes from the time of terminal sterilizing filtration. Longer intervals between sterilizing filtration and radiopharmaceutical administration are possible with proper validation that radiochemical and microbiological purity remain acceptable.

23. Before administration, visual inspection must confirm the final [⁶⁴Cu]Cu-MeOPTSM or [⁶⁴Cu]Cu-MeOETSM radiopharmaceutical product solution to be clear, colorless to faint yellow-brown, free of particulate matter, and free of cloudiness. At the lowest levels of carrier Cu, the solution is expected to be colorless. But, at the highest levels of carrier Cu allowed under the Curium specifications for the ⁶⁴Cu-chloride precursor, the final product may have a yellowish-brown tint. (The Cu-chelates are dark red in color.)

Routine quality control of [⁶⁴Cu]Cu-MeOPTSM: The following tests shall be completed as indicated on each batch of [⁶⁴Cu]Cu-MeOPTSM, prior to release for human administration.

A. Appearance: The [⁶⁴Cu]Cu-MeOPTSM drug product will be a transparent, colorless to faint yellow-brown, solution in acetate buffered saline containing ≤5% ethanol and ≤5% propylene glycol. It must not be used if visual inspection reveals a cloudy appearance or the presence of particulate matter. (The product solution will only have color at the highest allowed levels of carrier copper in Curium's specifications and CoA findings for the ⁶⁴Cu-chloride precursor. The Cu-MeOPTSM complex in bulk has a very dark red color. At extremely high dilution such solutions will appear yellowish-brown. For radiopharmaceutical batches made at the highest molar activity levels, the radiopharmaceutical product solution will likely be colorless on visual inspection.)

B. pH: The pH of the [⁶⁴Cu]Cu-MeOPTSM drug product shall be between 4.0 and 8.0.

C. Radionuclide Identity: Radionuclide identity is confirmed by the Certificate of Analysis that accompanies the starting ⁶⁴Cu chloride solution. In process validation, the identity is confirmed by repeated assays of an aliquot of the product using a radionuclide dose calibrator of established accuracy to establish the product's physical half-life. That assay shall result in a calculated half-life between 724.0 and 800.2 minutes (T_{1/2} of the ⁶⁴Cu radionuclide=762.1 minutes or 12.701 hours).

D. Radiochemical Purity: The radioactivity in the product be present as not less than 90% [⁶⁴Cu]Cu-MeOPTSM.

E. Membrane Filter Integrity: The integrity of the 0.2 μm sterilization filter shall be confirmed by a sterile filter integrity test. Prior to the release of product for human administration, the filter unit used for the terminal sterilizing filtration shall be tested to meet a bubble point specification of the filter manufacturer. A continuous stream of bubbles observed through the filter outlet at a pressure below the manufacturer's specification will indicate a ruptured or defective membrane filter (test failure). Test failure requires either rejection the [⁶⁴Cu]Cu-MeOPTSM batch, or reprocessing of the [⁶⁴Cu]Cu-MeOPTSM solution by repeating the sterilizing filtration through another sterile 0.2-μm filter unit, followed by confirmation that the second filter passes the bubble point test of its membrane integrity.

### Biological Quality Control of [⁶⁴Cu]Cu-MeOPTSM:

A. Bacterial Endotoxins: The final [⁶⁴Cu]Cu-MeOPTSM product formulation shall contain not more than 175 EU/V (USP endotoxin units), in which V is the maximum volume of human subject administration in mL.

B. Sterility: The final [⁶⁴Cu]Cu-MeOPTSM product formulation shall be sterile. Sterility testing shall be initiated within 30 hours of production

Labelling of the Radioactive Drug: The syringe containing the patient dose of the final product shall be labeled “[⁶⁴Cu]Cu-MeOPTSM Lot #⁶⁴Cu-MeOPTSM-YYYYMMDD-X.” This lot number will permit reference to the corresponding Batch Record Form which documents the suitability of the [⁶⁴Cu]Cu-MeOPTSM product for human use. The final product shall be stored at room temperature inside suitable radiation shielding, labeled: “[⁶⁴Cu]Cu-MeOPTSM; Lot #⁶⁴Cu-MeOPTSM-YYYYMMDD-X; RADIOACTIVE.

### Additional Embodiments

In another embodiment, a “cold kit” approach would be implemented for on-demand point-of-use radiopharmaceutical compounding with the desired copper radioisotope (⁶⁴Cu, ⁶¹Cu ⁶²Cu, ⁶⁰Cu, or ⁶⁷Cu), enabling convenient radiopharmaceutical compounding at, or near, the clinical site of use. In this embodiment, the bis(thiosemicarbazone) ligand would be provided in a sterile septum-capped vial, either as a lyophilized powder with suitable excipients, or as a solution with appropriate solvent(s) such as ethanol and/or propylene glycol. The desired Cu-radiopharmaceutical would be produced by aseptically adding the independently produced sterile radionuclide solution (^{60,61,62,64, or 67}Cu²⁺ in HCl or acetate-buffer) to the sterile vial containing the bis(thiosemicarbazone) ligand. The buffer could be added directly to the radiocopper before mixing with the bis(thiosemicarbazone) ligand, or the buffer could be pre-packages as an excipient with the bis(thiosemicarbazone) ligand awaiting direct addition of the radiocopper in an acid solution. A potential advantage of this kit-based synthesis approach is that the ligand kit vial could be centrally manufactured as a ready-to-use radiopharmaceutical precursor, and then broadly distributed to enable on-demand local/regional compounding of the radiopharmaceutical drug product by any nuclear pharmacy or medical facility with access to a supply of pharmaceutical grade radiocopper and standard facilities for compounding and dispensing of sterile radiopharmaceutical drug products.

In another embodiment, the ⁶⁴Cu or ⁶⁷Cu-labeled radiopharmaceutical would be centrally produced in a cGMP pharmaceutical manufacturing facility and distributed to the clinical user (or their local nuclear pharmacy) in either a sterile vial, or unit-dose syringe, with the supplied sterile product solution being ready for direct patient administration by the clinical end-user.

In another embodiment, the ⁶⁴Cu or ⁶¹Cu-labeled radiopharmaceutical would be regionally produced in a cGMP pharmaceutical manufacturing facility or radiopharmacy, and distributed to the clinical user in a unit-dose syringe ready for direct administration by the clinical end-user.

Table 1 provides predicted physicochemical properties of Cu-MeOPTSM, and related known copper (II) bis(thiosemicarbazone) radiopharmaceuticals. The Cu-MeOPTSM radiopharmaceutical is expected to satisfy all the stated design criteria for an improved novel agent for clinical development:

TABLE 1
Predicted properties of the proposed Cu-MeOPTSM radiopharmaceutical
and related agents.

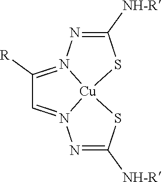

| | | | | | Brain | | Strong |
|---|---|---|---|---|---|---|---|
| | | | | | Uptake | Brain | Association |
| | | | | | in Rat at | Uptake in | with |
| | | | | | 1-min | Rat at 2- | Warfarin- |
| | | | | | Post- | hr Post- | binding |
| | | | Molecular | | injection | injection | Site of |
| Compound | R | R′ | Weight | Log P^{a} | (% ID) | (% ID) | HSA |
| Cu-PTSM | CH₃- | —CH₃ | 308 | 1.92 | 3.0 ± 0.7 | 3.2 ± 0.4 | Yes |
| Cu-ETS | CH₃CH₂- | —H | 294 | 1.35 | 0.71 ± 0.07 | 0.86 ± 0.08 | No |
| Cu-ETSM | CH₃CH₂- | —CH₃ | 322 | 2.65 | 2.8 ± 0.4 | 2.9 ± 0.3 | No |
| Cu-KTSM | CH₃CH₂OCH(CH₃)- | —CH₃ | 366 | 2.64 | 2.5 ± 0.5 | 2.5 ± 0.3 | No |
| Cu- | CH₃OCH₂- | —CH₃ | 338 | 1.6 | | | No |
| MeOPTSM | | | | (estimated) | | | (Predicted) |
| Cu- | CH₃OCH₂CH₂- | —CH₃ | 352 | 1.9 | | | No |
| MeOETSM | | | | (estimated) | | | (Predicted) |
| Cu-(3- | CH₃CH(OH)- * | —CH₃ | 338 | 1.7 | | | No |
| HO)ETSM | | | | (estimated) | | | (Predicted) |
| Cu-(4- | HOCH₂CH₂- | —CH₃ | 338 | 1.5 | | | No |
| HO)ETSM | | | | (estimated) | | | (Predicted) |
| Cu-cycloPr- | Cyclopropyl- | —CH₃ | 334 | 2.6 | | | No |
| GTSM | | | | (estimated) | | | (Predicted) |
| Cu- | Cyclobutyl- | —CH₃ | 348 | 3.0 | | | No |
| cycloBu- | | | | (estimated) | | | (Predicted) |
| GTSM | | | | | | | |
| Cu- | Cyclopentyl- | —CH₃ | 362 | 3.4 | | | No |
| cycloPen- | | | | (estimated) | | | (Predicted) |
| GTSM | | | | | | | |

*Introduces a chiral center, which may be undesirable, although the remaining chelate properties seem appropriate.

Cu-MeOPTSM is uncharged and lipophilic, with a predicted log P=1.6.

Cu-MeOPTSM is predicted to retain the good blood-brain barrier penetration seen with Cu-PTSM and Cu-ETSM, based on retention of the N4-methyl substituents of the Cu-PTSM and Cu-ETSM radiopharmaceuticals (Table 1, R′ substituents).

Cu-MeOPTSM is predicted to retain the susceptibility of Cu-PTSM, Cu-ETS, and Cu-ETSM to intracellular reductive decomposition and trapping of the radiocopper, thereby giving the high residual fraction desired for convenient and robust imaging following high first-pass tissue extraction. (The lower lipophilicity of Cu-MeOPTSM, relative to Cu-ETSM, may result in better intracellular trapping by reducing the tracer's residence time in cell membranes.)

Cu-MeOPTSM is predicted to avoid association with the Warfarin binding site of HSA, as do all studied tracers with substitution more bulky than a methyl substituent at the “R” position of the ketoaldehyde backbone (Table 1).

### Example 2—X-Ray Structure of Cu-MeOPTSM

A bulk sample of Cu(II)-MeOPTSM was prepared by reaction of the H₂MeOPTSM ligand (0.37 g) with bis(acetylacetonato)copper(II) (“Cu(acac)₂”) (0.35 g) in hot ethanol, with dark red crystals of the Cu(II)-MeOPTSM product subsequently deposited from the supersaturated solution that resulted upon cooling. Analysis of a sample of the Cu(II)-MeOPTSM product using high resolution positive ion electrospray ionization mass spectrometry shows the expected [m+H]⁺ parent ion [C₈H₁₅⁶³CuN₆O³²S₂] at m/z=338.0034 (expected m/z=338.0039).

For crystallographic structure determination the sample, a red, plate-like crystal (approximate dimensions 0.320×0.101× 0.022 mm³), was placed onto the tip of a MiTeGen loop and mounted on a Bruker Venture D8 diffractometer equipped with a PhotonIII detector at 123 (2) K.

### Data Collection

The data collection was carried out using Mo Kα radiation (graphite monochromator) with a frame time of 85, 60, and 5 seconds and a detector distance of 4.00 cm. A collection strategy was calculated and complete data to a resolution of 0.77 Å (eight sets of frames) were collected with 1° @ and φ scans. A total of 1860 frames were collected. The total exposure time was 19.29 hours. The frames were integrated with the Bruker SAINT software package1 using a narrow-frame algorithm. The integration of the data using a monoclinic unit cell yielded a total of 45433 reflections to a maximum θ angle of 27.58° (0.77 Å resolution), of which 3367 were independent (average redundancy 13.494, completeness=99.7%, Rᵢₙₜ=9.20%, R_{sig}=4.35%) and 2949 (87.59%) were greater than 2σ(F²). The final cell constants of a=15.6093(12) Å, b=3.9833(3) Å, c=23.5907(18) Å, β=96.289(3) °, volume=1457.96(19) Å3, are based upon the refinement of the XYZ-centroids of 9872 reflections above 20 σ(I) with 5.251°<2θ<54.98°. Data were corrected for absorption effects using the Multi-Scan method (SADABS2). The ratio of minimum to maximum apparent transmission was 0.706. The calculated minimum and maximum transmission coefficients (based on crystal size) are 0.5990 and 0.9620. Table 2 contains additional crystal and refinement information.

### Structure Solution and Refinement

The space group P2₁/n was determined based on intensity statistics and systematic absences. The structure was solved and refined using the SHELX suite of programs. An intrinsic-methods solution was calculated, which provided most non-hydrogen atoms from the E-map. Full-matrix least squares/difference Fourier cycles were performed, which located the remaining non-hydrogen atoms. All non-hydrogen atoms were refined with anisotropic displacement parameters. The hydrogen atoms were placed in ideal positions and refined as riding atoms with relative isotropic displacement parameters with exception of H10 and H2O (both connected to nitrogen), which were refined freely for all parameters. Disorder was refined for the C17-O18-C19 moiety (alternative site C27-O28-C19) using restraints and constraints. The structure contains solvent-accessible areas along the crystallographic b-axis in which solvent ethanol is disordered along a channel and over a special position. The solvent was partially and refined with a site occupancy set to 25%.

The final anisotropic full-matrix least-squares refinement on F² with 266 variables converged at R1=5.35%, for the observed data and wR2=11.84% for all data. The goodness-of-fit was 1.017. The largest peak in the final difference electron density synthesis was 0.184 e⁻/Å3 and the largest hole was −0.241 e⁻/Å³ with an RMS deviation of 0.050 e⁻/Å3. On the basis of the final model, the calculated density was 1.165 g/cm³ and F(000), 840 e.

TABLE 2
Crystal data and structure refinement for CuMeOPTSM.

| Empirical formula | C8.50 H15.50 Cu N6 O1.25 S2 | |
|---|---|---|
| Formula weight | 349.43 | |
| Crystal color, shape, size | red plate, 0.320 × 0.101 × 0.022 mm³ | |
| Temperature | 123(2) K | |
| Wavelength | 0.71073 Å | |
| Crystal system, space group | Monoclinic, P2₁/n | |
| Unit cell dimensions | a = 15.6093(12) Å | α = 90°. |
| | b = 3.9833(3) Å | β = 96.289(3)°. |
| | c = 23.5907(18) Å | γ = 90°. |

| | |
|---|---|
| Volume | 1457.96(19) Å³ |
| Z | 4 |
| Density (calculated) | 1.592 Mg/m³ |
| Absorption coefficient | 1.786 mm⁻¹ |
| F(000) | 718 |
| Data collection | |
| Diffractometer | Venture D8, Bruker |
| Source | Iμ3.0, Incoatec |
| Detector | Photon III |
| Theta range for data collection | 2.626 to 27.581°. |
| Index ranges | −20 <= h <= 20, −5 <= k <= 5, −30 <= l <= 30 |
| Reflections collected | 45433 |
| Independent reflections | 3367 [Rint = 0.0920] |
| Observed Reflections | 2949 |
| Completeness to theta = 25.242° | 99.9% |
| Solution and Refinement | |
| Absorption correction | Semi-empirical from equivalents |
| Max. and min. transmission | 0.7456 and 0.5262 |
| Solution | Intrinsic methods |
| Refinement method | Full-matrix least-squares on F2 |
| Weighting scheme | w = [σ²Fo² + AP² + BP]⁻¹, with |
| | P = (Fo² + 2 Fc²)/3, A = 0.0642, B = 2.2246 |
| Data/restraints/parameters | 3367/186/202 |
| Goodness-of-fit on F² | 1.126 |
| Final R indices [I > 2 □(I)] | R1 = 0.0484, wR2 = 0.1260 |
| R indices (all data) | R1 = 0.0568, wR2 = 0.1320 |
| Extinction coefficient | 0.0149(16) |
| Largest diff. peak and hole | 0.777 and −0.543 e.Å⁻³ |

Goodness-of-fit = [Σ[w(Fₒ² − F_{c}²)²]/N_{observns} − Nₚₐᵣₐₘₛ)]^{1/2}, all data.
R1 = Σ(|Fₒ| − |F_{c}|)/Σ |Fₒ|.
wR2 = [Σ[w(Fₒ² − F_{c}²)²]/Σ [w(Fₒ²)²]]^{1/2}.

TABLE 3
Atomic coordinates (×10⁴) and equivalent isotropic displacement
parameters (Å² × 10³) for CuMeOPTSM. U_{eq} is defined as
one third of the trace of the orthogonalized U^{ij} tensor.

| | x | y | z | U(eq) |
|---|---|---|---|---|
| Cu1 | 4611(1) | 4857(1) | 3191(1) | 26(1) |
| S1 | 3625(1) | 858(2) | 2904(1) | 27(1) |
| S2 | 4628(1) | 5734(2) | 4132(1) | 27(1) |
| N10 | 3138(2) | −9(7) | 1810(1) | 32(1) |
| N12 | 4370(2) | 3148(7) | 1968(1) | 31(1) |
| N13 | 4882(2) | 4641(7) | 2399(1) | 29(1) |
| N20 | 5952(2) | 8651(8) | 4731(1) | 31(1) |
| N22 | 6099(2) | 8492(7) | 3779(1) | 30(1) |
| N23 | 5707(2) | 7342(7) | 3274(1) | 28(1) |
| C11 | 3731(2) | 1441(9) | 2174(1) | 28(1) |
| C15 | 3125(3) | 208(11) | 1198(2) | 47(1) |
| C21 | 5632(2) | 7749(8) | 4204(1) | 27(1) |
| C25 | 6779(2) | 10248(10) | 4862(2) | 38(1) |
| C14 | 5568(2) | 6299(9) | 2299(2) | 32(1) |
| C17 | 5857(3) | 6746(13) | 1736(2) | 40(1) |
| O18 | 6629(3) | 8506(14) | 1762(2) | 62(1) |
| C19 | 6973(5) | 8650(20) | 1230(3) | 66(2) |
| C24 | 6051(2) | 7786(9) | 2802(2) | 33(1) |
| C27 | 6883(10) | 9100(40) | 2685(7) | 40(1) |
| O28 | 7267(9) | 7640(40) | 2239(6) | 62(1) |
| C29 | 7169(17) | 9580(60) | 1727(9) | 66(2) |
| O1S | 5180(20) | 8060(60) | −100(12) | 127(7) |
| C1S | 4740(20) | 5150(70) | 80(20) | 127(7) |
| C2S | 5260(30) | 2180(60) | −78(19) | 127(7) |

TABLE 4
Bond lengths [Å] and angles [°] for CuMeOPTSM.

| | | | |
|---|---|---|---|
| Cu1—N13 | 1.963(3) | Cu1—N23 | 1.967(3) |
| Cu1—S2 | 2.2433(9) | Cu1—S1 | 2.2668(9) |
| S1—C11 | 1.765(3) | S2—C21 | 1.752(3) |
| N10—C11 | 1.324(5) | N10—C15 | 1.443(5) |
| N10—H10 | 0.88(4) | N12—C11 | 1.341(4) |
| N12—N13 | 1.360(4) | N13—C14 | 1.301(4) |
| N20—C21 | 1.337(4) | N20—C25 | 1.442(5) |
| N20—H20 | 0.83(6) | N22—C21 | 1.334(4) |
| N22—N23 | 1.359(4) | N23—C24 | 1.300(4) |
| C15—H15A | 0.9800 | C15—H15B | 0.9800 |
| C15—H15C | 0.9800 | C25—H25A | 0.9800 |
| C25—H25B | 0.9800 | C25—H25C | 0.9800 |
| C14—C24 | 1.458(5) | C14—C17 | 1.461(6) |
| C14—H14 | 0.9500 | C17—O18 | 1.390(6) |
| C17—H17A | 0.9900 | C17—H17B | 0.9900 |
| O18—C19 | 1.420(7) | C19—H19A | 0.9800 |
| C19—H19B | 0.9800 | C19—H19C | 0.9800 |
| C24—C27 | 1.456(13) | C24—H24 | 0.9500 |
| C27—O28 | 1.394(15) | C27—O28#1 | 1.93(2) |
| C27—H27A | 0.9900 | C27—H27B | 0.9900 |
| O28—C29 | 1.428(17) | C29—H29A | 0.9800 |
| C29—H29B | 0.9800 | C29—H29C | 0.9800 |
| O1S—C1S | 1.434(10) | O1S—H10 | 0.8400 |
| C1S—C2S | 1.502(10) | C1S—H1S | 0.9900 |
| C1S—H1P | 0.9900 | C2S—H2S | 0.9800 |
| C2S—H2P | 0.9800 | C2S—H2U | 0.9800 |
| N13—Cu1—N23 | 80.81(12) | N13—Cu1—S2 | 165.51(9) |
| N23—Cu1—S2 | 84.74(9) | N13—Cu1—S1 | 83.97(9) |
| N23—Cu1—S1 | 159.71(9) | S2—Cu1—S1 | 109.80(3) |
| C11—S1—Cu1 | 93.78(12) | C21—S2—Cu1 | 94.60(12) |
| C11—N10—C15 | 123.7(3) | C11—N10—H10 | 116(2) |
| C15—N10—H10 | 120(2) | C11—N12—N13 | 110.6(3) |
| C14—N13—N12 | 121.0(3) | C14—N13—Cu1 | 114.6(3) |
| N12—N13—Cu1 | 124.1(2) | C21—N20—C25 | 123.5(3) |
| C21—N20—H20 | 119(4) | C25—N20—H20 | 117(4) |
| C21—N22—N23 | 110.7(3) | C24—N23—N22 | 121.2(3) |
| C24—N23—Cu1 | 114.8(2) | N22—N23—Cu1 | 124.0(2) |
| N10—C11—N12 | 118.8(3) | N10—C11—S1 | 116.2(3) |
| N12—C11—S1 | 125.0(3) | N10—C15—H15A | 109.5 |
| N10—C15—H15B | 109.5 | H15A—C15—H15B | 109.5 |
| N10—C15—H15C | 109.5 | H15A—C15—H15C | 109.5 |
| H15B—C15—H15C | 109.5 | N22—C21—N20 | 117.3(3) |
| N22—C21—S2 | 125.8(3) | N20—C21—S2 | 116.8(3) |
| N20—C25—H25A | 109.5 | N20—C25—H25B | 109.5 |
| H25A—C25—H25B | 109.5 | N20—C25—H25C | 109.5 |
| H25A—C25—H25C | 109.5 | H25B—C25—H25C | 109.5 |
| N13—C14—C24 | 114.9(3) | N13—C14—C17 | 124.6(4) |
| C24—C14—C17 | 120.4(3) | N13—C14—H14 | 122.5 |
| C24—C14—H14 | 122.5 | O18—C17—C14 | 111.9(4) |
| O18—C17—H17A | 109.2 | C14—C17—H17A | 109.2 |
| O18—C17—H17B | 109.2 | C14—C17—H17B | 109.2 |
| H17A—C17—H17B | 107.9 | C17—O18—C19 | 113.0(5) |
| O18—C19—H19A | 109.5 | O18—C19—H19B | 109.5 |
| H19A—C19—H19B | 109.5 | O18—C19—H19C | 109.5 |
| H19A—C19—H19C | 109.5 | H19B—C19—H19C | 109.5 |
| N23—C24—C27 | 132.2(7) | N23—C24—C14 | 114.5(3) |
| C27—C24—C14 | 112.7(7) | N23—C24—H24 | 122.7 |
| C14—C24—H24 | 122.7 | O28—C27—C24 | 117.2(12) |
| C24—C27—O28#1 | 149.8(12) | O28—C27—H27A | 108.0 |
| C24—C27—H27A | 108.0 | O28#1—C27—H27A | 50.5 |
| O28—C27—H27B | 108.0 | C24—C27—H27B | 108.0 |
| O28#1—C27—H27B | 67.6 | H27A—C27—H27B | 107.2 |
| C27—O28—C29 | 113.3(16) | C27—O28—C27#2 | 125.7(11) |
| C29—O28—C27#2 | 118.9(14) | O28—C29—H29A | 109.5 |
| O28—C29—H29B | 109.5 | H29A—C29—H29B | 109.5 |
| O28—C29—H29C | 109.5 | H29A—C29—H29C | 109.5 |
| H29B—C29—H29C | 109.5 | C1S—O1S—H10 | 109.5 |
| O1S—C1S—C2S | 105.8(13) | O1S—C1S—H1S | 110.6 |
| C2S—C1S—H1S | 110.6 | O1S—C1S—H1P | 110.6 |
| C2S—C1S—H1P | 110.6 | H1S—C1S—H1P | 108.7 |
| C1S—C2S—H2S | 109.3 | C1S—C2S—H2P | 110.1 |
| H2S—C2S—H2P | 109.5 | C1S—C2S—H2U | 109.0 |
| H2S—C2S—H2U | 109.5 | H2P—C2S—H2U | 109.5 |

Symmetry transformations used to generate equivalent atoms:
#1 −x + 3/2, y + 1/2, −z + 1/2
#2 −x + 3/2, y − 1/2, −z + 1/2

TABLE 5
Anisotropic displacement parameters (Å² × 10³) for CuMeOPTSM.
The anisotropic displacement factor exponent takes the
form: −2π²[h² a*²U¹¹ + . . . + 2 h k a* b* U¹²].

| | U¹¹ | U²² | U³³ | U²³ | U¹³ | U¹² |
|---|---|---|---|---|---|---|
| Cu1 | 25(1) | 26(1) | 29(1) | 2(1) | 10(1) | −1(1) |
| S1 | 27(1) | 24(1) | 31(1) | 2(1) | 9(1) | −1(1) |
| S2 | 27(1) | 27(1) | 29(1) | 3(1) | 7(1) | −2(1) |
| N10 | 34(2) | 30(2) | 32(2) | 2(1) | 5(1) | −2(1) |
| N12 | 35(1) | 27(1) | 32(1) | 1(1) | 11(1) | 1(1) |
| N13 | 30(1) | 25(1) | 34(2) | 2(1) | 12(1) | 2(1) |
| N20 | 30(1) | 29(2) | 33(2) | 1(1) | 4(1) | 1(1) |
| N22 | 24(1) | 29(1) | 36(1) | 1(1) | 7(1) | 0(1) |
| N23 | 25(1) | 27(1) | 35(1) | 3(1) | 9(1) | 3(1) |
| C11 | 28(1) | 25(2) | 32(2) | 3(1) | 8(1) | 5(1) |
| C15 | 60(3) | 46(2) | 34(2) | −2(2) | 2(2) | −7(2) |
| C21 | 28(1) | 20(1) | 36(2) | 2(1) | 6(1) | 6(1) |
| C25 | 31(2) | 38(2) | 45(2) | −6(2) | 2(2) | −1(2) |
| C14 | 31(2) | 32(2) | 37(2) | 4(1) | 15(1) | 1(1) |
| C17 | 42(2) | 39(2) | 42(2) | −1(2) | 19(2) | −9(2) |
| O18 | 54(2) | 84(3) | 52(2) | −4(2) | 24(2) | −23(2) |
| C19 | 65(4) | 82(4) | 56(3) | 1(3) | 30(3) | −19(3) |
| C24 | 29(2) | 31(2) | 40(2) | 5(1) | 14(1) | −1(1) |
| C27 | 42(2) | 39(2) | 42(2) | −1(2) | 19(2) | −9(2) |
| O28 | 54(2) | 84(3) | 52(2) | −4(2) | 24(2) | −23(2) |
| C29 | 65(4) | 82(4) | 56(3) | 1(3) | 30(3) | −19(3) |
| O1S | 121(9) | 153(10) | 109(8) | 6(8) | 18(7) | −3(8) |
| C1S | 121(9) | 153(10) | 109(8) | 6(8) | 18(7) | −3(8) |
| C2S | 121(9) | 153(10) | 109(8) | 6(8) | 18(7) | −3(8) |

TABLE 6
Hydrogen coordinates (×10⁴) and isotropic displacement
parameters (Å² × 10³) for CuMeOPTSM.

| | x | y | z | Ueq |
|---|---|---|---|---|
| H10 | 2730(20) | −1100(100) | 1963(15) | 24(9) |
| H20 | 5640(30) | 8490(160) | 4990(20) | 72(18) |
| H15A | 2566 | −571 | 1016 | 70 |
| H15B | 3584 | −1201 | 1074 | 70 |
| H15C | 3216 | 2544 | 1089 | 70 |
| H25A | 6843 | 10989 | 5260 | 58 |
| H25B | 6818 | 12194 | 4612 | 58 |
| H25C | 7238 | 8648 | 4802 | 58 |
| H14 | 5741 | 6525 | 1928 | 48 |
| H17A | 5932 | 4517 | 1562 | 59 |
| H17B | 5409 | 7976 | 1488 | 59 |
| H19A | 7598 | 8330 | 1290 | 99 |
| H19B | 6844 | 10845 | 1053 | 99 |
| H19C | 6713 | 6878 | 979 | 99 |
| H24 | 6574 | 8983 | 2785 | 49 |
| H27A | 6814 | 11532 | 2603 | 59 |
| H27B | 7287 | 8883 | 3037 | 59 |
| H29A | 7543 | 8674 | 1457 | 99 |
| H29B | 7328 | 11918 | 1816 | 99 |
| H29C | 6567 | 9487 | 1558 | 99 |
| H10 | 5182 | 8028 | −456 | 191 |
| HIS | 4149 | 5050 | −119 | 191 |
| H1P | 4709 | 5216 | 495 | 191 |
| H2S | 5232 | 2026 | −494 | 191 |
| H2P | 5030 | 119 | 74 | 191 |
| H2U | 5860 | 2485 | 84 | 191 |

TABLE 7
Torsion angles [°] for CuMeOPTSM.

| | | | |
|---|---|---|---|
| C11—N12—N13—C14 | 176.7(3) | C11—N12—N13—Cu1 | −9.2(4) |
| C21—N22—N23—C24 | −179.9(3) | C21—N22—N23—Cu1 | −1.0(4) |
| C15—N10—C11—N12 | −1.6(5) | C15—N10—C11—S1 | 179.9(3) |
| N13—N12—C11—N10 | 176.0(3) | N13—N12—C11—S1 | −5.7(4) |
| Cu1—S1—C11—N10 | −167.8(3) | Cu1—S1—C11—N12 | 13.9(3) |
| N23—N22—C21—N20 | 178.2(3) | N23—N22—C21—S2 | −2.6(4) |
| C25—N20—C21—N22 | −2.4(5) | C25—N20—C21—S2 | 178.3(3) |
| Cu1—S2—C21—N22 | 4.0(3) | Cu1—S2—C21—N20 | −176.7(2) |
| N12—N13—C14—C24 | 179.5(3) | Cu1—N13—C14—C24 | 4.8(4) |
| N12—N13—C14—C17 | 0.1(6) | Cu1—N13—C14—C17 | −174.6(3) |
| N13—C14—C17—O18 | −177.4(4) | C24—C14—C17—O18 | 3.2(6) |
| C14—C17—O18—C19 | 173.4(5) | N22—N23—C24—C27 | 7.2(12) |
| Cu1—N23—C24—C27 | −171.8(10) | N22—N23—C24—C14 | 177.8(3) |
| Cu1—N23—C24—C14 | −1.2(4) | N13—C14—C24—N23 | −2.4(5) |
| C17—C14—C24—N23 | 177.0(4) | N13—C14—C24—C27 | 170.0(9) |
| N23—C24—C27—O28 | 144.0(11) | C14—C24—C27—O28 | −26.7(18) |
| N23—C24—C27—O28#1 | −54(3) | C14—C24—C27—O28#1 | 135(2) |
| C24—C27—O28—C29 | 101.2(19) | O28#1—C27—O28—C29 | −69.7(15) |
| C24—C27—O28—C27#2 | −95.7(13) | O28#1—C27—O28—C27#2 | 93.4(18) |

Symmetry transformations used to generate equivalent atoms:
#1 −x + 3/2, y + 1/2, −z + 1/2
#2 −x + 3/2, y − 1/2, −z + 1/2

TABLE 8
Hydrogen bonds for CuMeOPTSM [Å and °].

| D-H . . . A | d(D-H) | d(H . . . A) | d(D . . . A) | <(DHA) |
|---|---|---|---|---|
| C29\hat{ }b-H29B\hat{ }b . . . O28\hat{ }b#1 | 0.98 | 2.27 | 2.78(3) | 111.5 |
| N10—H10 . . . S1#3 | 0.88(4) | 2.48(4) | 3.338(3) | 163(3) |
| N20—H20 . . . S2#4 | 0.83(6) | 2.73(6) | 3.407(3) | 140(5) |

Symmetry transformations used to generate equivalent atoms:
#1 −x + 3/2, y + 1/2, −z + 1/2
#2 −x + 3/2, y − 1/2, −z + 1/2
#3 −x + 1/2, y − 1/2, −z + 1/2
#4 −x + 1, −y + 1, −z + 1

### Example 3-Formulation of the [⁶⁴Cu]Cu-MeOPTSM

The composition and results of a representative formulation of [⁶⁴Cu]Cu-MeOPTSM are provided in Tables 9-11.

TABLE 9
Formulation of the [⁶⁴Cu]Cu-MeOPTSM Radiopharmaceutical Drug Product.

| Component | Quantity | |
|---|---|---|
| Sodium Chloride for Injection | 5 | mL |
| 0.25M Ultrapure Sodium Acetate | 0.5 | mL (0.125 mmol NaOAc) |
| Sterile Water for Injection | 0.3 | mL |
| Ethanol (USP) | 0.3 | mL |
| Propylene Glycol (USP) | 0.3 | mL |
| 0.2% HCl (0.055M) | 0.10-0.16 | mL (0.0055-0.0088 mmol HCl) |
| H₂MeOPTSM | 15-μg (0.054 μmol) | |
| ⁶⁴Cu | 70-110 | mCi* |

*At manufacturer calibration (06:00 CT the day prior to actual synthesis, formulation, and use).

TABLE 10
Composition and Purity Standards for the
[⁶⁴Cu]Cu-MeOPTSM Radiopharmaceutical Drug Product.

| | |
|---|---|
| Targeted Administered Radioactivity: | 8-17 mCi |
| Expiration Time: | 60-minutes from sterilization by terminal |
| | filtration. |
| Solution Composition: | Acetate-buffered sodium chloride for |
| | injection with 5% ethanol and 5% propylene |
| | glycol. |
| | (Expected administered volume: ~5-mL) |
| Final Product Container: | Septum-capped sterile glass vial |
| Final Patient Dose Container: | Single-Use Sterile Luer-Lock Syringe |
| | Closed by a Single-Use Sterile Cap |

Release Specifications

| | |
|---|---|
| Appearance: | Clear with no visual evidence of cloudiness |
| | or particulates. |
| Radionuclide Identity and Purity: | CoA Conforms to Curium Specifications |
| Radiochemical Purity: | ≥90% |
| Endotoxin Level: | <175 EU per dose |
| pH: | 4.0-8.0 |
| Bubble Point of PVDF Sterilization Filter: | ≥50 psig |
| USP Sterility Test (Retrospective): | PASS |

Additional Specifications for ValidationDoses

| | |
|---|---|
| Radiochemical Purity (>1 hour post- | ≥90% |
| filtration): | |
| Appearance at >1 hour post-filtration: | Clear with no visual evidence of cloudiness |
| | or particulates. |
| Radionuclidic Identity and Purity: | Measured physical half-life = 12.1-13.3 |
| | hours |

TABLE 11
Test Results for Five Consecutive Sample Batches of [⁶⁴Cu]Cu-MeOPTSM.

| | Date Prepared: | | | | | | | | | |
|---|---|---|---|---|---|---|---|---|---|---|
| | 6 Sep. 2024 | | 13 Sep. 2024 | | 20 Sep. 2024 | | 25 Oct. 2024 | | 01 Nov. 2024 | |
| | Lot Number: | | | | | | | | | |
| | 64Cu- | | 64Cu- | | 64Cu- | | 64Cu- | | 64Cu- | |
| | MeOPTSM- | | MeOPTSM- | | MeOPTSM- | | MeOPTSM- | | MeOPTSM- | |
| | 20240906-1 | | 20240913-1 | | 20240920-1 | | 20241025-1 | | 20241101-1 | |
| Starting ⁶⁴Cu: | 110 | mCi ^{†} | 110 | mCi ^{†} | 110 | mCi ^{†} | 110 | mCi ^{†} | 110 | mCi ^{†} |
| Cu content of | 2 | μg/mL | 21 | μg/mL | 17 | μg/mL | 7 | μg/mL | 9 | μg/mL |
| ⁶⁴Cu chloride | | | | | | | | | | |
| (spec. ≤30 | | | | | | | | | | |
| μg/mL) ^{††} | | | | | | | | | | |
| Volume of | 0.176 | mL | 0.154 | mL | 0.161 | mL | 0.177 | mL | 0.163 | |
| ⁶⁴Cu-chloride ^{††} | | | | | | | | | | |
| H₂MeOPTSM | 15 | μg | 15 | μg | 15 | μg | 15 | μg | 15 | μg |
| in Synthesis | | | | | | | | | | |
| Radionuclide | CoA | | CoA | | CoA | | CoA | | CoA | |
| Identity and | Conforms to | | Conforms to | | Conforms to | | Conforms to | | Conforms to | |
| Purity: | Specifications | | Specifications | | Specifications | | Specifications | | Specifications | |
| Intermediate | 23.4 | mCi | 21.8 | mCi | 25.7 | mCi | 20.6 | mCi | 21.8 | mCi |
| Product Assay | | | | | | | | | | |
| (before | | | | | | | | | | |
| filtration): | | | | | | | | | | |
| Intermediate | 13:21 | | 12:14 | | 11:51:00 | | 12:12:00 | | 12:03:55 | |
| Product Assay | | | | | | | | | | |
| Time: | | | | | | | | | | |
| Nominal | 6.0 | mL | 6 | mL | 6 | mL | 6 | mL | 6 | mL |
| Product | | | | | | | | | | |
| Volume: | | | | | | | | | | |
| Expiration | 14:23 | | 13:15 | | 12:53 | | 13:16 | | 13:10 | |
| Time: | | | | | | | | | | |
| ^{‡} ‘Sample | 16.80 | mCi | 14.47 | mCi | 18.92 | mCi | 15.51 | mCi | 17.74 | mCi |
| Dose’ Assay: | | | | | | | | | | |
| Sample Dose | 13:42:00 | | 12:29:00 | | 12:06:00 | | 12:30:00 | | 12:23:00 | |
| Assay Time: | | | | | | | | | | |
| ^{‡} Sample Dose | 5.0 | mL | 5.0 | mL | 5.0 | mL | 5.0 | mL | 5.0 | mL |
| Volume: | | | | | | | | | | |
| Radiochemical | 97.8% | | 92.1% | | 97.8% | | 97.9% | | 98.8% | |
| Purity (≥90%): | | | | | | | | | | |
| Endotoxin | <5.00 | | <5.00 | | <5.00 | | <5.00 | | <5.00 | |
| Level | EU/mL | | EU/mL | | EU/mL | | EU/mL | | EU/mL | |
| (<175 | | | | | | | | | | |
| EU/dose): | | | | | | | | | | |
| pH (4.0-8.0): | 5.5 | | 5.2 | | 5.5 | | 5.5 | | 5.5 | |
| Bubble Point | 59 | psig | 59 | psig | 58 | psig | 60 | psig | 59 | psig |
| of Sterilization | | | | | | | | | | |
| Filter (50 psig | | | | | | | | | | |
| minimum): | | | | | | | | | | |

| | | | | | |
|---|---|---|---|---|---|
| Appearance: | PASSED | PASSED | PASSED | PASSED | PASSED |
| Sterility | PASSED | PASSED | PASSED | PASSED | PASSED |
| (Retrospective): | | | | | |

Additional Testing for Validation Batches

| Radiochemical | 98.2% | | 93.4% | | 97.9% | | 96.5% | | 98.1% | |
|---|---|---|---|---|---|---|---|---|---|---|
| Purity After | | | | | | | | | | |
| Expiration | | | | | | | | | | |
| (≥90%): | | | | | | | | | | |
| Appearance | PASSED | | PASSED | | PASSED | | PASSED | | PASSED | |
| After | | | | | | | | | | |
| Expiration: | | | | | | | | | | |
| Radionuclide | 12.9 | hr | 12.5 | hr | 12.8 | hr | 12.7 | hr | 12.7 | hr |
| Identity | | | | | | | | | | |
| Confirmation | | | | | | | | | | |
| (measured | | | | | | | | | | |
| half-life = | | | | | | | | | | |
| 12.1-13.3 | | | | | | | | | | |
| hours): | | | | | | | | | | |

*Complete Batch Record Forms are attached for reference.
^{†} Radioactivity at shipment calibration time, 06:00 CT the day prior to synthesis.
^{‡} During validations, sample doses were drawn at 5.0 mL volume regardless of final activity.
^{††} From Curium Certificate of Analysis

### Example 4

Additional copper chelate compounds containing a cycloalkyl moiety may also be utilized in imaging methods to deliver the desired imaging properties. Scheme 3 outlines the proposed synthesis of exemplary compounds containing a cyclopropyl, cyclobutyl, or cyclopentyl. “Cu” may be a copper radioisotope selected from the group consisting of ⁶⁰C, ⁶¹Cu, ⁶²Cu, ⁶⁴Cu, and ⁶⁷Cu, or a stable (nonradioactive) isotope of Cu selected from the group consisting of ⁶³C and ⁶⁵Cu.

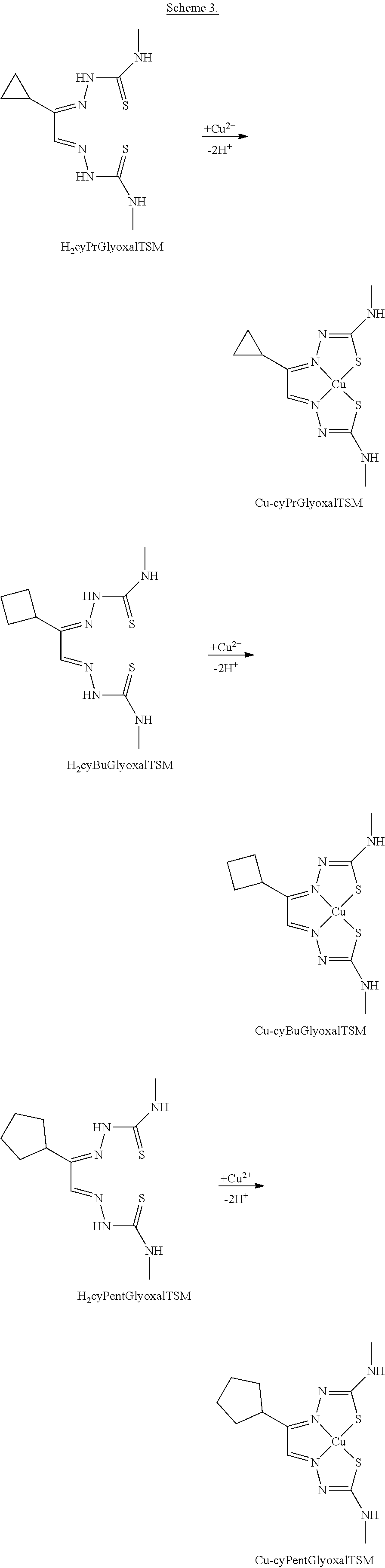

### REFERENCES

E K John and M A Green, Structure-activity relationships for metal-labeled blood flow tracers: comparison of keto aldehyde bis(thiosemicarbazonato)copper(II) derivatives, J Med Chem 33:1764-1770; 1990.
C J Mathias, et al., Evaluation of a potential generator-produced PET tracer for cerebral perfusion imaging: single-pass cerebral extraction measurements and imaging with radiolabeled Cu-PTSM, J Nucl Med 31:351-359; 1990 (documents single-pass cerebral extraction and retention of Cu-PTSM and Cu-ETSM in baboons).
C J Mathias, S R Bergmann, and M A Green, Species-dependent binding of copper(II) bis(thiosemicarbazone) radiopharmaceuticals to serum albumin, J Nucl Med 36:1451-1456; 1995. NE Basken, C J Mathias, A E Lipka, M A Green, Species dependence of [⁶⁴Cu]Cu-bis(thiosemicarbazone) radiopharmaceutical binding to serum albumins, Nucl Med Biol, 35:281-286, 2008.
N E Basken, C J Mathias, and M A Green, Elucidation of the human serum albumin (HSA) binding site for the Cu-PTSM and Cu-ATSM radiopharmaceuticals, J Pharm Sci, 98:2170-2179, 2009.
N E Basken and M A Green, Cu(II)Bis(thiosemicarbazone) radiopharmaceutical binding to serum albumin: further definition of species-dependent and associated substituent effects, Nucl Med Biol, 36:495-504, 2009.
C. J. Mathias, W. H. Margenau, J. W. Brodack, M. J. Welch, and M. A. Green. A Remote System for the Synthesis of Copper-62 Labeled Cu(PTSM), Applied Radiation and Isotopes, 42, 317-320, 1991.
M. A. Green, C. J. Mathias, M. J. Welch, F. Fernandez-Rubio, J. S. Perlmutter, M. E. Raichle, and S. R. Bergmann. [⁶²Cu]-Labeled Pyruvaldehyde Bis(N4-methylthiosemicarbazonato)-copper (II): Synthesis and Evaluation as a Positron Emission Tomography Tracer for Cerebral and Myocardial Perfusion, J. Nucl. Med., 31, 1989-1996, 1990.
J. W. Fletcher, T. F. Logan, J. A. Eitel, C. J. Mathias, Y. Ng, J. L. Lacy, G. D. Hutchins, and M. A. Green. Whole-body PET/CT Evaluation of Tumor Perfusion using Generator-based [⁶²Cu]Cu-ETS: Validation by Direct Comparison to [¹⁵O]Water in Metastatic Renal Cell Carcinoma. J Nucl Med 56:56-62; 2015.
USP<823> Positron Emission Tomography Drugs for Compounding, Investigational, and research Uses. USP—NF. United States Pharmacopeial Convention, 2017.
SAINT V8.40A (2020), Bruker AXS, Madison, WI
L. Krause, R. Herbst-Irmer, G. M. Sheldrick, D. Stalke: Comparison of silver and molybdenum microfocus X-ray sources for single-crystal structure determination. J. Appl. Cryst., 48, 3-10 (2015). doi:10.1107/S1600576714022985.
G. M. Sheldrick: SHELXT—Integrated space-group and crystal-structure determination. Acta Cryst. A71, 3-8 (2015). doi:10.1107/S2053273314026370.
G. M. Sheldrick: Crystal structure refinement with SHELXL. Acta Cryst. C71, 3-8 (2015). doi:10.1107/S2053229614024218.

1. A compound of formula (I), or a pharmaceutically acceptable salt thereof, 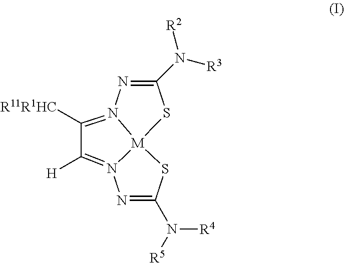
wherein
(i) R¹ is R^{A}—O—(CH₂)ₙ and R¹¹ is H or C₁₋₆alkyl; or
(ii) R¹ and R¹¹ together with the carbon atom they are attached to form a C₃₋₆cycloalkyl;
and
R², R³, R⁴, R⁵ are independently H or C₁₋₆alkyl;
R^{A} is H or C₁₋₆alkyl;
n is 0, 1, 2, 3, 4, 5, or 6; and
M is a copper isotope;
provided that R¹R¹¹CH— is not Me(EtO)CH.

2. The compound of claim 1,
wherein
R¹ is R^{A}—O—(CH₂)ₙ;
R¹¹, R², R³, R⁴, R⁵ are independently H or C₁₋₆alkyl;
R^{A} is H or C₁₋₆alkyl;
n is 0, 1, 2, 3, 4, 5, or 6; and
M is a copper radioisotope selected from the group consisting of ⁶⁰C, ⁶¹Cu, ⁶²Cu, ⁶⁴Cu, and ⁶⁷Cu,
provided that R¹R¹¹CH— is not Me(EtO)CH.

3. The compound of claim 2, or a pharmaceutically acceptable salt thereof, wherein R¹¹ is H.

4. The compound of claim 2, or a pharmaceutically acceptable salt thereof, wherein n is 0 or 1.

5. The compound of claim 2, or a pharmaceutically acceptable salt thereof, wherein R^{A} is H or methyl.

6. The compound of claim 2, or a pharmaceutically acceptable salt thereof, wherein R¹ is MeO—, MeOCH₂—, or HOCH₂—; and R¹¹ is H.

7. The compound of claim 2, or a pharmaceutically acceptable salt thereof, wherein R² and R⁴ are H; and R³ and R⁵ are C₁₋₆alkyl.

8. The compound of claim 2, or a pharmaceutically acceptable salt thereof, wherein R¹ is MeO— or MeOCH₂—; R¹¹ is H; R² and R⁴ are H; and R³ and R⁵ are methyl.

9. The compound of claim 2, or a pharmaceutically acceptable salt thereof, wherein M is ⁶²Cu or ⁶⁴Cu.

10. The compound of claim 1, or a pharmaceutically acceptable salt thereof, wherein R¹ and R¹¹ together with the carbon atom they are attached to form a cyclopropyl, cyclobutyl, or cyclopentyl.

11. The compound of claim 1, or a pharmaceutically acceptable salt thereof, which is

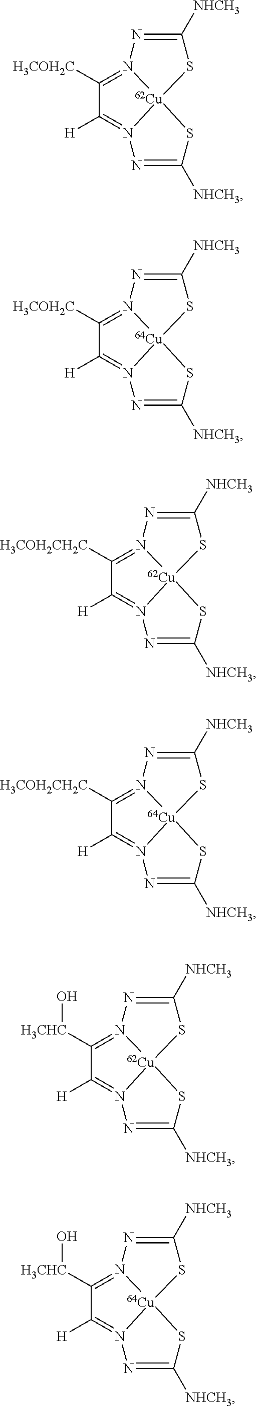

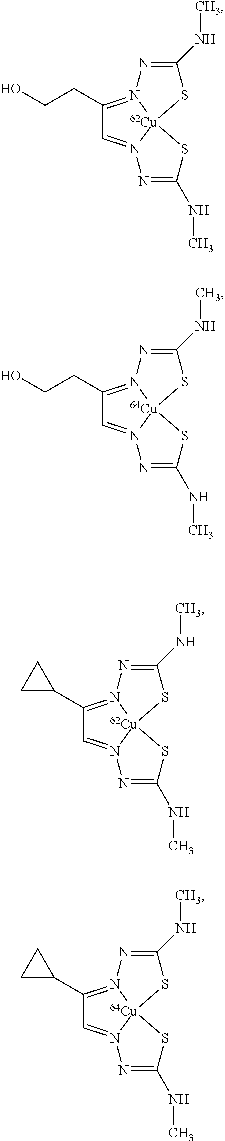

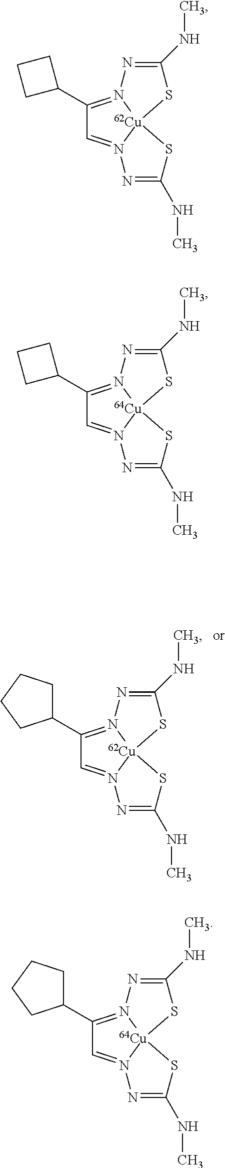

12. A pharmaceutical composition comprising the compound of claim 1, or a pharmaceutically acceptable salt thereof, and a pharmaceutically acceptable carrier.

13. A method of imaging a subject, the method comprising:
administering the pharmaceutical composition of claim 12 to the subject; and
acquiring a signal from the copper isotope in the subject, thereby generating an image of the subject,
wherein the copper isotope is a copper radioisotope selected from the group consisting of ⁶⁰C, ⁶¹Cu, ⁶²Cu, ⁶⁴Cu, and ⁶⁷Cu.

14. The method of claim 13, wherein the signal is acquired by positron emission tomography (PET).

15. The method of claim 13, wherein the pharmaceutical composition is administered to the subject via intravenous administration.

16. The method of claim 13, wherein administering the pharmaceutical composition to the subject comprises perfusing a tissue of the subject with the compound of formula (I), or a pharmaceutically acceptable salt thereof; and wherein the signal from the perfused tissue is acquired.

17. The method of claim 13, wherein the image is generated from whole body of the subject, optionally further comprising quantitatively mapping the signal from one or more perfused tissues of the subject.

18. The method of claim 13, further comprising evaluating a physiological condition of the subject based on the image, optionally wherein the physiological condition comprises myocardial blood flow, cerebral blood flow, renal blood flow, tumor blood flow, musculoskeletal blood flow, other organ blood flow, or a combination thereof.

19. (canceled)

20. A method of performing positron emission tomography (PET) imaging of a subject, the method comprising:
administering the pharmaceutical composition of claim 12 to the subject; and
acquiring a signal from the copper isotope in the subject by a radiation detector, thereby generating an image of the subject,
wherein the copper isotope is a copper radioisotope selected from the group consisting of ⁶⁰C, ⁶¹Cu, ⁶²Cu, ⁶⁴Cu, and ⁶⁷Cu.

21. A method of identifying tissue-specific distribution of a radiolabeled complex comprising a radiocopper in a subject, the method comprising imaging an excised tissue having the radiocopper therein.

22. (canceled)

23. (canceled)
